(19) United States
(12) Patent Application Publication
(10) Pub. No.: US 2015/0150224 A1
(43) Pub. Date: Jun. 4, 2015

(54) TRANSGENIC MOUSE MODELS FOR MC4R

(71) Applicant: UNIVERSITE DE MONTREAL, Montreal (CA)

(72) Inventors: Michel Bouvier, Montreal (CA); Patricia Rene, Montreal (CA); Benjamin Turgeon, Montreal (CA)

(73) Assignee: UNIVERSITE DE MONTREAL, Montreal, QC (CA)

(21) Appl. No.: 14/407,779

(22) PCT Filed: Jun. 14, 2013
(86) PCT No.: PCT/CA2013/050457

§ 371 Date: Dec. 12, 2014

Related U.S. Application Data
(60) Provisional application No. 61/659,739, filed on Jun. 14, 2012.

(51) Int. Cl. A01K 67/027; C12N 15/90; G01N 33/50; C07K 14/72; A61K 49/00

(52) U.S. Cl. CPC A01K 67/0276; C07K 14/72; A61K 49/0008; G01N 33/5023; C12N 15/907; A01K 2267/0362; A01K 2227/105; C12N 2015/8527

(57) ABSTRACT

There are provided herein transgenic non-human animals and cells comprising a transgene encoding either a mutated human melanocortin type-4 receptor (hMC4R) protein, wherein the mutated protein is misfolded and retained intracellularly, or a wild-type human melanocortin type-4 receptor (hMC4R) protein. Transgenes and targeting constructs used to produce such transgenic animals and cells are also provided, as well as methods for using the transgenic animals in pharmaceutical screening and as commercial research animals for modeling obesity.

### FIELD OF THE INVENTION

The present invention relates to knock-in mouse models for human melanocortin type-4 receptor. The invention provides transgenic non-human animals and transgenic non-human mammalian cells harboring a transgene encoding an obesity-causing mutant form of human melanocortin type-4 receptor (hMC4R), e.g., comprising the R165W mutation, or wild-type hMC4R, and further comprising functionally disrupted endogenous MC4R loci. There are also provided transgenes and targeting constructs used to produce such transgenic cells and animals, transgenes encoding human obesity-causing mutant MC4R polypeptide sequences and human wild-type MC4R polypeptide sequences, and methods for using the transgenic animals in pharmaceutical screening and as commercial research animals for modeling obesity.

### BACKGROUND OF THE INVENTION

Disease-causing mutations in G protein-coupled receptors often lead to decreased cell surface expression and concomitant loss of function as a result of improper folding. These mutant receptors, generally recognized by the cell's quality control system within the endoplasmic reticulum and Golgi apparatus, are retained intracellularly and targeted for degradation. In many of these conformational diseases, the mutation occurs in receptor domains that do not directly affect ligand binding or G protein coupling, opening the possibility for interventions that could restore receptor function by rescuing folding and cell surface expression.

The melanocortin-4 receptor (MC4R) plays a pivotal role in energy homeostasis (Cone, R. D., 2005, Nat. Neurosci., 8:571-578). In humans, MC4R mutations lead to an obese phenotype similar to the homozygous null-mouse model (Huszar et al., 1997, Cell, 88:131-141; Chen et al., 2000, Transgenic Res., 9:145-154). Heterozygous null mutations in the melanocortin-4 receptor (MC4R) cause early-onset obesity in humans and are the most common cause of monogenic obesity to date (Farooqi, S. and O'Rahilly, S., Endocr Rev 27, 710-718 (2006)). More than 150 distinct mutations of MC4R have been reported in the obese human population. Intracellular retention of the receptor has been proposed to be the most frequent consequence of the mutations, with more than 50% of childhood obesity-related MC4R mutations leading to partial or complete retention in the biosynthetic pathway via the quality control system acting on misfolded receptors (Wang, Z. Q. and Tao, Y. X., Biochimica et Biophysica Acta, 1812, 1190-1199 (2011)).

Recovering cell surface expression of intracellularly-retained mutant forms of MC4R could therefore have a beneficial therapeutic value. This idea was tested using a pharmacological chaperone approach (Rene, P. et al., 2010, J. Pharmacol. Exp. Ther., 335:520-532). Five distinct cell-permeant selective ligands were used to restore cell surface targeting and function of nine different mutant forms of human MC4R (hMC4R) found in obese patients. Treatment with any of the five compounds led to total or partial restoration of cell surface expression (assessed by flow cytometry) and signaling activity (assessed by measuring cAMP accumulation upon agonist stimulation), with efficacy dependent upon the MC4R mutant form and compound being tested. These findings indicated that pharmacological chaperones represent candidates for the development of a targeted therapy suitable for patients with MC4R-linked early-onset obesity.

Current strategies to control early-onset obesity are either modestly effective or highly invasive (e.g., bariatric surgery). Lifestyle modification, such as diet and exercise or current therapeutics are modestly effective in maintaining long-term weight loss in patients with class III (BMI>40 kg/m²) obesity. Bariatric surgery is currently the most effective therapy for these patients, but outcomes after bariatric surgery are variable. Moreover, lifestyle interventions based on exercise, behavior, and nutrition therapy in children with MC4R mutations are not effective in providing long-term weight loss in contrast to children without MC4R mutations. The success of bariatric surgery for children carrying MC4R mutations is also unpredictable and often ineffective in the long-term (Asian, I. R. et al., International Journal of Obesity, (2011), 35, 457-461, Hatoum, I. J. et al., J. Clin. Endocrinol. Metab., (2012), 97(6):0000-0000). Development of alternative therapeutic approaches is therefore necessary, especially for MC4R-induced obesity.

A clinically active pharmacological chaperone would represent an attractive therapeutic avenue. However, in order to test a pharmacological chaperone preclinically, it would be beneficial to have a mouse model. Currently available MC4R transgenic models are knock-out models, i.e., the MC4R gene is removed. These models do not allow assessment of therapeutic candidates acting as pharmacological chaperones, which require models producing misfolded receptors that are retained intracellularly. Current MC4R transgenic models also do not allow study or direct visualization of the MC4 receptor, e.g., visualization of the receptor's localization. There is a need therefore for a mouse model of MC4R-linked obesity, which recapitulates phenotypic features of patients with MC4R-deficiency and allows testing of therapeutic approaches for MC4R-deficiency, and for obesity in general.

### SUMMARY OF THE INVENTION

In an aspect of the invention, there is provided herein a knock-in transgenic mouse model expressing an obesity-causing mutant form of human MC4R (hMC4R) in the mouse locus for melanocortin 4 receptor (mMC4R). In the knock-in mouse model, the mouse MC4R gene is replaced with a mutated human MC4R gene, creating humanized MC4R mice. In an embodiment, the mutation in the mutated human MC4R gene causes misfolding of the melanocortin 4 receptor and intracellular retention of the receptor, thereby preventing the receptor from being expressed at the cell surface and abolishing receptor function. This leads to an obese phenotype in humans (Farooqi, S. and O'Rahilly, S., Endocr. Rev. 27, 710-718 (2006); Wang, Z. Q. and Tao, Y. X., Biochimica et Biophysica Acta, 1812, 1190-1199 (2011)). In one embodiment, the mutation in the mutated human MC4R gene is the R165W mutation (Nijenhuis, W. A. et al., J. Biol. Chem. 278, 22939-22945 (2003)).

In an aspect of the invention, there are provided non-human animals harboring at least one copy of a transgene comprising a polynucleotide sequence which encodes a heterologous MC4R polypeptide comprising a mutation, e.g., the R165W mutation, operably linked to a transcription regulatory sequence capable of producing expression of the heterologous MC4R polypeptide in the transgenic non-human animal. Said heterologous MC4R polypeptide comprising a mutation generally is expressed in cells which normally express the naturally-occurring endogenous MC4R gene (if present). Typically, the non-human animal is a mouse and the heterologous MC4R gene is a human R165W mutation MC4R gene. Such transgenes typically comprise a R165W mutation MC4R coding sequence. In an embodiment, a transgene comprises a promoter and optionally an enhancer, which is linked to and drives expression of structural sequences encoding a heterologous MC4R polypeptide comprising a R165W mutation. In another embodiment, a transgene comprises a R165W mutation MC4R coding sequence under control of the endogenous mMC4R promoter. In an embodiment, non-human animals provided herein are homozygous for a transgene of the invention. In another embodiment, non-human animals provided herein are heterozygous for a transgene of the invention.

In another aspect, the invention provides transgenes comprising a gene encoding a mutated hMC4R, e.g., a human R165W mutation MC4R gene, said gene operably linked to a transcription regulatory sequence functional in the host transgenic animal. Such transgenes are typically integrated into a host chromosomal location by homologous integration. The transgenes may further comprise a selectable marker, such as a neo or gpt gene operably linked to a constitutive promoter, such as a phosphoglycerate kinase (pgk) promoter or HSV tk gene promoter linked to an enhancer (e.g., SV40 enhancer). Transgenes may further comprise markers or tags such as fluorescent proteins, e.g., yellow fluorescent protein, or HA. Selectable markers or tags may also be flanked by sites allowing use of site-specific recombinase systems (e.g., FLP-FRT, Cre-Lox, etc.) to remove the marker or tag sequences after integration into a host chromosomal location.

The invention further provides non-human transgenic animals, typically non-human mammals such as mice, which harbor at least one copy of a transgene or targeting construct of the invention, either homologously or nonhomologously integrated into an endogenous chromosomal location so as to encode a mutated MC4R polypeptide, e.g., a R165W mutation MC4R polypeptide. Such transgenic animals are usually produced by introducing the transgene or targeting construct into a fertilized egg or embryonic stem (ES) cell, typically by microinjection, electroporation, lipofection, or biolistics. The transgenic animals express the R165W mutation MC4R gene of the transgene (or homologously recombined targeting construct). Such animals are suitable for use in a variety of disease models and drug screening uses, as well as other applications.

In an embodiment, a transgene or targeting construct is homologously integrated into a non-human transgenic animal, i.e., integration is targeted and a “knock-in” animal is made. In another embodiment, a transgene or targeting construct is nonhomologously integrated, i.e., integration is not targeted.

The invention also provides non-human animals and cells which harbor at least one integrated targeting construct that encodes a mutated hMC4R polypeptide, e.g., hMC4R having a mutation that causes misfolding of the polypeptide and intracellular retention thereof, e.g., hMC4R comprising the R165W mutation.

The invention also provides transgenic non-human animals, such as non-primate mammals, e.g., rodents, e.g., mice, that have at least one inactivated endogenous MC4R allele, and preferably are homozygous for inactivated endogenous MC4R alleles, and which are substantially incapable of directing the efficient expression of endogenous (i.e., wildtype) MC4R. For example, in an embodiment, a transgenic mouse is homozygous for inactivated endogenous MC4R alleles and is substantially incapable of producing murine MC4R encoded by an endogenous (i.e., naturally-occurring) MC4R gene. Such a transgenic mouse, having inactivated endogenous MC4R genes, is a preferred host recipient for a transgene encoding a heterologous MC4R polypeptide, e.g., a human mutated MC4R polypeptide, e.g., a human R165W mutation MC4R polypeptide. For example, human MC4R comprising the R165W mutation may be encoded and expressed from a heterologous transgene(s) in such transgenic mice. Such heterologous transgenes may be integrated in a nonhomologous location in a chromosome of the non-human animal, or may be integrated by homologous recombination or gene conversion into a non-human MC4R gene locus, thereby effecting simultaneous knockout of the endogenous MC4R gene (or segment thereof) and replacement with the human MC4R gene (or segment thereof).

In an embodiment, there are provided herein transgenic non-human animals and transgenic non-human mammalian cells harboring a transgene encoding a MC4R polypeptide comprising the R165W mutation. The transgene encoding a MC4R polypeptide comprising the R165W mutation may be homologously or non homologously integrated.

In an aspect of the invention, there is provided herein a knock-in transgenic mouse model expressing a wild-type form of human MC4R (hMC4R) in the mouse locus for melanocortin 4 receptor (mMC4R). In this knock-in mouse model, the mouse MC4R gene is replaced with a wild-type human MC4R gene, creating humanized MC4R mice. In an embodiment, there are provided non-human animals harboring at least one copy of a transgene comprising a polynucleotide sequence which encodes a wild-type human MC4R polypeptide, operably linked to a transcription regulatory sequence capable of producing expression of the human MC4R polypeptide in the transgenic non-human animal. Said human MC4R polypeptide generally is expressed in cells, which normally express the naturally-occurring endogenous MC4R gene (if present). Typically, the non-human animal is a mouse. In an embodiment, a transgene comprises a promoter and optionally an enhancer, which is linked to and drives expression of structural sequences encoding a wild-type human MC4R polypeptide. In another embodiment, a transgene comprises a wild-type human MC4R coding sequence under control of the endogenous mMC4R promoter. In an embodiment, non-human animals provided herein are homozygous for a transgene of the invention. In another embodiment, non-human animals provided herein are heterozygous for a transgene of the invention. Such transgenes are typically integrated into a host chromosomal location by homologous integration. The transgenes may further comprise a selectable marker, such as a neo or gpt gene operably linked to a constitutive promoter, such as a phosphoglycerate kinase (pgk) promoter or HSV tk gene promoter linked to an enhancer (e.g., SV40 enhancer). Transgenes may further comprise markers or tags such as fluorescent proteins, e.g., yellow fluorescent protein, myc or HA. Selectable markers or tags may also be flanked by sites allowing use of site-specific recombinase systems (e.g., FLP-FRT, Cre-Lox, etc.) to remove the marker or tag sequences after integration into a host chromosomal location.

The invention further provides non-human transgenic animals, typically non-human mammals such as mice, which harbor at least one copy of a transgene or targeting construct of the invention, either homologously or nonhomologously integrated into an endogenous chromosomal location so as to encode a wild-type human MC4R polypeptide. Such transgenic animals are usually produced by introducing the transgene or targeting construct into a fertilized egg or embryonic stem (ES) cell, typically by microinjection, electroporation, lipofection, or biolistics. The transgenic animals express the wild-type human MC4R gene of the transgene (or homologously recombined targeting construct).

The invention also provides non-human animals and cells, which harbor at least one integrated targeting construct that encodes a wild-type human MC4R polypeptide (hMC4R). The invention further provides transgenic non-human animals, such as non-primate mammals, e.g., rodents, e.g., mice, that have at least one inactivated endogenous MC4R allele, and preferably are homozygous for inactivated endogenous MC4R alleles, and which are substantially incapable of directing the efficient expression of endogenous (i.e., wildtype) mouse MC4R. For example, in an embodiment, a transgenic mouse is homozygous for inactivated endogenous MC4R alleles and is substantially incapable of producing murine MC4R encoded by an endogenous (i.e., naturally-occurring) MC4R gene. Such a transgenic mouse, having inactivated endogenous MC4R genes, is a preferred host recipient for a transgene encoding a heterologous MC4R polypeptide, e.g., a human wild-type MC4R polypeptide. For example, wild-type human MC4R may be encoded and expressed from a heterologous transgene(s) in such transgenic mice. Such heterologous transgenes may be integrated in a nonhomologous location in a chromosome of the non-human animal, or may be integrated by homologous recombination or gene conversion into a non-human MC4R gene locus, thereby effecting simultaneous knockout of the endogenous MC4R gene (or segment thereof) and replacement with the human MC4R gene (or segment thereof).

In an embodiment, there are provided herein transgenic non-human animals and transgenic non-human mammalian cells harboring a transgene encoding a wild-type human MC4R polypeptide. The transgene encoding a wild-type human MC4R polypeptide may be homologously or nonhomologously integrated.

In one embodiment, there is provided herein a transgenic non-human animal comprising in its genome a transgene encoding a mutated human melanocortin type-4 receptor (hMC4R) protein, wherein the mutated hMC4R protein promotes obesity. In an embodiment, the mutated hMC4R protein is non-functional. For example, the mutated hMC4R protein may be improperly folded compared to wild-type hMC4R protein and/or retained intracellularly. In one embodiment, a mutated hMC4R protein comprises an arginine at position 165 of the hMC4R protein in place of a tryptophan (R165W mutation).

In some embodiments, transgenic non-human animals provided herein are heterozygous for a mutated hMC4R protein. In some embodiments, transgenic non-human animals provided herein are homozygous for a mutated hMC4R protein. In an embodiment, the endogenous animal MC4R gene is functionally disrupted or deleted and replaced by a transgene encoding a mutated hMC4R protein.

Such a transgene may further comprise a detectable marker or tag, such as a fluorescent protein, a human influenza hemagglutinin (HA) tag, or a myc tag. A fluorescent protein may be, for example, green fluorescent protein, red fluorescent protein, blue fluorescent protein, cyan fluorescent protein, or yellow fluorescent protein. In an embodiment, yellow fluorescent protein is encoded by a Venus gene sequence. In some embodiments, a transgene is double-tagged with an HA tag and a fluorescent protein. In some embodiments, transgenes further comprise a site-specific recombinase system, such as FLP-FRT or Cre-Lox. In an embodiment, a transgene comprises a yellow fluorescent protein encoded by a Venus gene sequence, and the Venus gene sequence is flanked by LoxP sites, allowing removal of the yellow fluorescent protein in a transgenic non-human animal. In some embodiments, transgenes comprise a neomycin cassette flanked by FRT sites. In some embodiments, transgenes comprise a myc tag, e.g., at the N-terminus of a mutated hMC4R protein.

In some embodiments, a transgene is inserted into an animal genome via homologous recombination. Non-human transgenic animals of the invention may be mammals, e.g., rodents, e.g., mice.

In some embodiments, a transgenic non-human animal has symptoms of MC4R-induced obesity. Non-limiting examples of such symptoms include obesity, hyperphagia, increased fat mass, increased linear growth, and/or obesity-associated metabolic disorders, relative to a nontransgenic non-human animal. In some embodiments, a transgenic non-human animal of the invention can be used as a model of obesity or MC4R-deficiency.

In an aspect of the invention, there are provided herein transgenic non-human mammalian cells or tissues comprising a transgene encoding a mutated human melanocortin type-4 receptor (hMC4R) protein. In an embodiment, a mutated hMC4R protein is non-functional, e.g., a mutated hMC4R protein is improperly folded compared to wild-type hMC4R protein and/or is retained intracellularly. In one embodiment, a mutated hMC4R protein comprises an arginine at position 165 of the hMC4R protein in place of a tryptophan (R165W mutation).

In an embodiment, a transgenic non-human mammalian cell or tissue is heterozygous for a mutated hMC4R protein. In an embodiment, a transgenic non-human mammalian cell or tissue is homozygous for a mutated hMC4R protein. In an embodiment, the endogenous non-human mammalian MC4R gene is functionally disrupted or deleted and replaced by a transgene encoding a mutated hMC4R protein.

In an embodiment, a transgene is inserted into the genome of a cell or tissue via homologous recombination. In an embodiment, a mammalian cell is a rodent cell or a mammalian tissue is a rodent tissue. In an embodiment, a rodent cell or tissue is mouse cell or tissue.

In some embodiments, transgenes provided herein comprise a yellow fluorescent protein encoded by a Venus gene sequence, and the Venus gene sequence is flanked by LoxP sites. In some embodiments, transgenes provided herein comprise a neomycin cassette flanked by FRT sites. In some embodiments, transgenes comprise a myc tag and/or a HA tag. In some embodiments, transgenes comprise sequences for insertion into a host chromosome at the MC4R locus via homologous recombination.

In an aspect, there are provided herein targeting constructs comprising transgenes of the invention.

In an aspect, there are provided herein methods of screening for an agent for treating obesity or for treating MC4R deficiency. In an embodiment, there is provided a method of screening for an agent for treating obesity or for treating MC4R deficiency, comprising providing a transgenic non-human animal of the invention, wherein a transgene is expressed to produce a mutated human MC4R protein; administering an agent to the transgenic non-human animal; and determining level of obesity in the transgenic non-human animal; wherein a reduced level of obesity or obesity-associated metabolic disorders in the transgenic non-human animal compared to the level of obesity or obesity-associated metabolic disorders in a control transgenic non-human animal which is not administered the agent indicates the agent is for use for treating obesity. In an embodiment, the mutated hMC4R protein is misfolded and/or retained intracellularly. In an embodiment, cell surface expression and/or signaling activity of the mutated hMC4R protein are determined, wherein an increase in cell surface expression and/or signaling activity of the mutated hMC4R protein after treatment with the agent, compared to the control transgenic non-human animal, indicates that the agent is for use for treating obesity.

In an embodiment, there is provided a method of screening for a pharmacological chaperone compound, comprising: providing a transgenic non-human animal comprising a transgene encoding a mutated human melanocortin type-4 receptor (hMC4R) protein, wherein the mutated hMC4R protein promotes obesity, wherein the transgene is expressed to produce the mutated hMC4R protein; administering a test compound to the transgenic non-human animal; and determining cell surface expression and/or signaling activity of the mutated hMC4R protein in the transgenic non-human animal; wherein an increase in cell surface expression and/or signaling activity of the mutated hMC4R protein in the transgenic non-human animal compared to a control transgenic non-human animal which is not administered the test compound indicates the test compound is a pharmacological chaperone. In an embodiment, the mutated hMC4R protein comprises an arginine at position 165 of the hMC4R protein in place of a tryptophan (R165W mutation).

In an embodiment, there is provided a method of screening for a pharmacological chaperone compound in vitro, comprising providing a transgenic non-human mammalian cell or tissue of the invention, wherein the transgene is expressed to produce a mutated hMC4R protein; administering a test compound to the transgenic non-human mammalian cell or tissue; and determining cell surface expression and/or signaling activity of the mutated hMC4R protein in the transgenic non-human mammalian cell or tissue; wherein an increase in cell surface expression and/or signaling activity of the mutated hMC4R protein in the transgenic non-human mammalian cell or tissue compared to a control transgenic non-human mammalian cell or tissue which is not administered the test compound indicates the test compound is a pharmacological chaperone.

In an aspect, there is provided herein a transgenic non-human animal comprising in its genome a transgene encoding a wild-type human melanocortin type-4 receptor (hMC4R) protein. The animal may be heterozygous or homozygous for the wild-type hMC4R protein. In an embodiment, the endogenous animal MC4R gene is functionally disrupted or deleted and replaced by the transgene encoding the wild-type hMC4R protein. The transgene encoding a wild-type hMC4R protein may also comprise a detectable marker or tag, such as a fluorescent protein, a human influenza hemagglutinin (HA) tag, or a myc tag. The fluorescent protein may be, for example, green fluorescent protein, red fluorescent protein, blue fluorescent protein, cyan fluorescent protein, or yellow fluorescent protein. In an embodiment, yellow fluorescent protein is encoded by a Venus gene sequence. The transgene may be double-tagged with an HA tag and a fluorescent protein or a myc tag and a fluorescent protein. In an embodiment, the transgene encoding a wild-type hMC4R protein is myc-tagged, e.g., at the N-terminus of the wild-type hMC4R protein. In some embodiments, the transgene further comprises a site-specific recombinase system, such as FLP-FRT or Cre-Lox. In an embodiment, the transgene comprises a yellow fluorescent protein encoded by a Venus gene sequence, and the Venus gene sequence is flanked by LoxP sites, allowing removal of the yellow fluorescent protein in the transgenic non-human animal. In an embodiment, a fluorescent protein is located in frame with the MC4R coding sequence, at the C-terminus. In an embodiment, the transgene comprises a neomycin cassette flanked by FRT sites. In an embodiment, the transgene encoding a wild-type hMC4R protein is inserted into the animal genome via homologous recombination.

In an aspect, there is provided herein a non-human mammalian cell or tissue comprising in its genome a transgene encoding a wild-type human melanocortin type-4 receptor (hMC4R) protein. Transgenes encoding a wild-type human melanocortin type-4 receptor (hMC4R) protein are also provided, as well as targeting constructs comprising the transgenes.

In an aspect, there is provided herein a method of screening for a MC4R ligand in diet-induced obesity, comprising providing a transgenic non-human animal comprising a transgene encoding wild-type hMC4R protein, wherein the transgene is expressed to produce the wild-type human MC4R protein; administering an agent to the transgenic non-human animal; and determining level of obesity or obesity-associated metabolic disorders in the transgenic non-human animal; wherein a reduced level of obesity or obesity-associated metabolic disorders in the transgenic non-human animal compared to the level of obesity or obesity-associated metabolic disorders in a control transgenic non-human animal which is not administered the agent indicates the agent is a MC4R ligand. Similar methods may also be used to study MC4R, using transgenic non-human animals comprising a transgene encoding wild-type hMC4R protein, for example to determine changes in MC4R expression in diet-induced obesity or to monitor how MC4R ligands and other anti-obesity therapeutic drugs affect the melanocortin pathway.

In one embodiment, a mutated R165W MC4R protein of the invention comprises the amino acid sequence set forth in SEQ ID NO: 25 or SEQ ID NO: 27, or a sequence substantially identical thereto, or a variant thereof. In an embodiment, a wild-type MC4R protein of the invention comprises the amino acid sequence set forth in SEQ ID NO: 26 or SEQ ID NO: 28, or a sequence substantially identical thereto, or a variant thereof. Thus, in some embodiments of the invention, transgenic non-human animals, transgenic non-human cells, transgenic non-human mammalian cells, transgenic non-human tissues, and/or transgenic non-human mammalian tissues of the invention comprise a protein having the amino acid sequence set forth in SEQ ID NO: 25 or 27 (for R165W MC4R protein) or SEQ ID NO: 26 or 28 (for wild type MC4R protein), or a sequence substantially identical thereto, or a variant thereof.

in some embodiments of the invention, transgenic non-human animals, transgenic non-human cells, transgenic non-human mammalian cells, transgenic non-human tissues, and/or transgenic non-human mammalian tissues of the invention comprise a nucleic acid molecule encoding the amino acid sequence set forth in SEQ ID NO: 25 or 27 (for R165W MC4R protein) or SEQ ID NO: 26 or 28 (for wild type MC4R protein), or a sequence substantially identical thereto, or a variant thereof.

in some embodiments of the invention, transgenic non-human animals, transgenic non-human cells, transgenic non-human mammalian cells, transgenic non-human tissues, and/or transgenic non-human mammalian tissues of the invention comprise a nucleic acid molecule having the sequence set forth in SEQ ID NO: 19, 20, 21, 22, 23, 24, 29, or 30, or a sequence substantially identical thereto, or a variant thereof.

In other embodiments, transgenes and/or targeting constructs of the invention comprise a nucleic acid molecule encoding the amino acid sequence set forth in SEQ ID NO: 25 or 27 (for R165W MC4R protein) or SEQ ID NO: 26 or 28 (for wild type MC4R protein), or a sequence substantially identical thereto, or a variant thereof. In some embodiments, transgenes and/or targeting constructs of the invention comprise a nucleic acid molecule having the sequence set forth in SEQ ID NO: 19, 20, 21, 22, 23, 24, 29, or 30, or a sequence substantially identical thereto, or a variant thereof.

### BRIEF DESCRIPTION OF THE DRAWINGS

For a better understanding of the invention and to show more clearly how it may be carried into effect, reference will now be made by way of example to the accompanying drawings, which illustrate aspects and features according to embodiments of the present invention, and in which:

FIG. 1 shows a schematic diagram of the procedure used for generating the knock-in transgenic mouse of the invention.

FIG. 2 shows a schematic diagram of the targeting vector design strategy.

FIG. 3 shows results from screening ES clones containing the transgenic allele at the mMC4R locus. The Upper panel shows autoradiograms of a Southern blot analysis from ES genomic DNA. The Lower panel shows schematic diagrams of the restriction digest strategy to distinguish between transgenic and non-transgenic alleles.

FIG. 4 shows screening of ES clones containing the transgenic allele without the selective neomycin cassette at the mMC4R locus. The Upper panel shows a Southern blot analysis of genomic DNA from ES positive cells after excision of the Neomycin cassette and digestion with ApaI, Sac I, EcoRV/BamHI by using the DNA probe shown in the diagram in the lower panel (red thick bar). The Lower panel shows a schematic representation of the restriction digest strategy to distinguish between transgenic allele after neomycin cassette excision and non-transgenic allele at the mMC4R locus.

FIG. 5 shows screening for transgenic mice from transmission test with chimeric mice carrying the transgenic allele. The Upper panel shows a schematic diagram of the screening strategy. The Lower panel is a Southern blot analysis of SacI-digested tail genomic DNA by using the probe shown on the upper diagram (red thick bar).

FIG. 6 shows breeding of the R165W knock-in (KI) mouse line to obtain each genotype for phenotype characterization. The Upper panel shows a schematic diagram of the breeding of heterozygous R165W knock-in (KI) mice. The Lower panel shows a diagram illustrating the restriction digest strategy to distinguish between the transgenic and non-transgenic allele at the mMC4R locus and a Southern blot analysis of SacI-digested tail genomic DNA by using the probe shown on the upper diagram (red thick bar).

FIG. 7 shows phenotypic characterization of hMC4R (R165W) knock-in mice (18-22 week-old). (A) shows immunohistology of frozen brain from heterozygous hMC4R(R165W) mice confirming expression of the mutant allele, where 3HA-hMC4R (R165W)-Venus expressing neurons in the paraventricular nucleus (PVN) were labelled. (B) shows body weight curve of transgenic and non-transgenic (non-Tg) littermates. (C) shows average food intake (F.I.) measured during Dark phase (Dark F.I.) and Light phase (Light F.I.), (D) shows fat mass measurements in grams. (E) shows snout-anus length for an example (shown in picture). The histograms show the snout-anus length in centimeters. Data are expressed as Mean±SEM. Numbers of animal per genotype are indicated in brackets.

FIG. 8 shows preliminary phenotypic characterization of hMC4R (WT) knock-in mice (17-24 week-old). (A) shows immunohistology of frozen brain from heterozygous hMC4R(WT) mice confirming expression of the WI allele, where myc-hMC4R (WT)-Venus expressing neurons in the paraventricular nucleus (PVN) were labelled. (B) shows body weight curve of transgenic and non-transgenic (non-Tg) littermates and the histograms show weight gain at 20 week-old. (C) shows daily average food intake (F.I.) and shows fat mass measurements in grams. (D) shows snout-anus length. The histograms show the snout-anus length in centimeters. Data are expressed as Mean±SEM. Numbers of animal per genotype are indicated in brackets.

FIG. 9 shows food intake and weight loss measurements upon DCPMP treatment in hMC4R (R165W) male mice. Mice were intraperitoneally injected one dose daily with vehicle or DCPMP at 30 mg per kg one hour before light off, for 9 days. Data are the average daily food intake (A, B, C), or the mean of the total body weight loss [total body weight before treatment—total body weight after 9 days treatment] (D), for each experimental group. Data are expressed as Mean±SEM. Numbers of animal per genotype are indicated in brackets.

FIG. 10 shows the nucleotide sequence of a 3HA-hMC4R(R165W)-Venus KI construct used in exemplary methods described herein, wherein: 3HA tag is shown in lower case (position +3275-3358):GTG is the first amino acid of MC4R, coding for Valine; the mutation R165W is shown hi lower case (position +3648); the ApaI site mutated is shown in lower case (position +4128-4133); the end of MC4R coding sequence is at position +4349, coding for Y; the ATG Venus coding sequence is shown in lower case (position +4394-4396); and the sequence left after excision of the Neo cassette by FRT recombination is indicated as FRT site in lower case (position +5154-5188). The nucleotide sequence shown here (SEC) ID NO: 23) is the nucleotide sequence of the transgene present in ES cells, before injection into blastocysts, i.e., the sequence wherein the neomycin cassette has been excised, and the sequence present at the mMC4R locus in mice exemplified herein.

FIG. 11 shows the nucleotide sequence of a myc-hMC4R(WT)-Venus KI construct used in exemplary methods described herein, wherein: myc tag is shown in lower case (position +3272-3306); GIG is the first amino acid of MC4R, coding for Valine; the Apa I site mutated is shown at position +4077; the end of the MC4R coding sequence is shown at position +4298, coding for Y; the ATG Venus coding sequence is shown in lower case (position +4343); and the sequence left after excision of the Neo cassette by FRT recombination is indicated as FRT site in lower case (position +5103-5138). The nucleotide sequence shown here (SEC) ID NO: 24) is the nucleotide sequence of the transgene present in ES cells, before injection into blastocysts, i.e., the sequence wherein the neomycin cassette has been excised, and the sequence present at the mMC4R locus in mice exemplified herein.

FIG. 12 shows the DNA sequence of a 3HA-hMC4R(R165W)-Venus KI construct without neomycin cassette (SE) ID NOs: 23, 29) and the corresponding protein sequence (SEC) ID NO: 27).

FIG. 13 shows the DNA sequence of a myc-hMC4R(WT)-Venus KI construct without neomycin cassette (SEC) ID NOs: 24, 30) and the corresponding protein sequence (SEQ ID NO: 28).

### DETAILED DESCRIPTION

We report herein the development of a unique transgenic mouse model for genetic obesity. This mouse model is a “knock-in” mouse line expressing an obesity-causing mutant form of the human MC4R (hMC4R) in the receptor's mouse locus, thus replacing the mouse gene and creating humanized MC4R mice. In an embodiment, the obesity-causing mutant form of hMC4R in the knock-in carries a R165W mutation. This mutation was selected because of its prevalence in humans and because of its capacity to be efficiently restored functionally by pharmacological chaperone (PC) compounds in cellular systems (Rene, P. et al., 2010, J. Pharmacol. Exp. Ther., 335:520-532). We also report herein the development of humanized transgenic mouse models expressing wild-type human MC4R in the receptor's mouse locus.

The mouse model provided herein represents the first knock-in mouse model of MC4R-induced obesity and provides several advantages over previously available MC4R mouse models. Previous models were primarily MC4R knock-out models, which removed the MC4R gene or prevented its expression, and thus did not allow visualization of the MC4 receptor protein, physiological studies of MC4R, or testing of MC4R-targeting compounds, in contrast to mouse models provided herein (Huszar, D. et al., Cell, 88, 131-141, (1997); Balthasar, N. et al., Cell, 123, 493-505, (2005)). Non-limiting examples of possible uses or advantages for mouse models provided herein include: a) visualization of MC4R protein and/or physiological studies of MC4R; b) testing physiological and/or potential therapeutic action of candidate MC4R PC compounds; c) testing other therapeutic approaches for treating MC4R-linked obesity, e.g., testing efficacy of small molecule therapeutic candidates, establishing pre-clinical proof of principle for new therapeutics targeting MC4R-deficiency and/or obesity in general; d) detection of the transgene in situ by double-labeling immunohistochemistry, e.g., using direct fluorescence; e) ability to follow the maturation and fate of MC4R receptor in situ; f) ability to study MC4R-induced obesity, especially in comparison to other forms of obesity, as well as therapeutics therefor; g) allowing a better understanding of the role of MC4R in physiological processes other than energy homeostasis, such as bone metabolism or inflammation; h) use for ex vivo studies of hMC4R from explants, cells, or slices of tissue from the mice; i) use for molecular studies of hMC4R in situ; and j) use to produce new mouse models by crossing or breeding the mice with other mouse strains, transgenic or otherwise. In addition, since mouse models provided herein express the human form of MC4R, they may be more relevant for human pharmacology profiling than previous mouse models. Mouse models provided herein are expected to provide at least one of the above uses or advantages. It is also noted that knock-in humanized wild-type MC4R mouse models may have additional advantages or uses, such as use to screen for MC4 ligands in diet-induced obesity or other pathological conditions such as cachexia.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below:

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The terms “substantially corresponds to”, “substantially homologous” or “substantial identity” as used herein denote a characteristic of a nucleic acid sequence, wherein a nucleic acid sequence has at least 70 percent sequence identity as compared to a reference sequence, typically at least 85 percent sequence identity, at least 90 percent sequence identity, at least 95 percent sequence identity, or at least 98 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 18 nucleotides long, typically at least 30 nucleotides long, at least 50 nucleotides long, or at least 100 nucleotides long. “Substantially complementary” as used herein refers to a sequence that is complementary to a sequence that substantially corresponds to a reference sequence.

Specific hybridization is defined herein as the formation of hybrids between a targeting transgene sequence (e.g., a polynucleotide of the invention which may include substitutions, deletion, and/or additions) and a specific target DNA sequence (e.g., a human MC4R gene sequence), wherein a labeled targeting transgene sequence preferentially hybridizes to the target such that, for example, a single band corresponding to a restriction fragment of a gene can be identified on a Southern blot of DNA prepared from cells using said labeled targeting transgene sequence as a probe. It is evident that optimal hybridization conditions will vary depending upon the sequence composition and length(s) of the targeting transgene(s) and endogenous target(s), and the experimental method selected by the practitioner. Various guidelines may be used to select appropriate hybridization conditions (see, Maniatis et al., Molecular Cloning: A Laboratory Manual (1989), 2nd Ed., Cold Spring Harbor, N.Y. and Berger and Kimmel, Methods in Enzymology, Volume 152, Guide to Molecular Cloning Techniques (1987), Academic Press, Inc., San Diego, Calif., which are incorporated herein by reference).

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by humans in the laboratory is naturally-occurring. As used herein, laboratory strains of rodents which may have been selectively bred according to classical genetics are considered naturally-occurring animals.

The term “cognate” as used herein refers to a gene sequence that is evolutionarily and functionally related between species. For example but not limitation, in the human genome, the human immunoglobulin heavy chain gene locus is the cognate gene to the mouse immunoglobulin heavy chain gene locus, since the sequences and structures of these two genes indicate that they are highly homologous and both genes encode a protein which functions to bind antigens specifically.

As used herein, the term “xenogenic” is defined in relation to a recipient mammalian host cell or non-human animal and means that an amino acid sequence or polynucleotide sequence is not encoded by or present in, respectively, the naturally-occurring genome of the recipient mammalian host cell or non-human animal. Xenogenic DNA sequences are foreign DNA sequences; for example, a human MC4R gene is xenogenic with respect to murine ES cells; also, for illustration, a human cystic fibrosis-associated CFTR allele is xenogenic with respect to a human cell line that is homozygous for wild-type (normal or non-mutated) CFTR alleles. Thus, a cloned murine nucleic acid sequence that has been mutated (e.g., by site directed mutagenesis) is xenogenic with respect to the murine genome from which the sequence was originally derived, if the mutated sequence does not naturally occur in the murine genome.

As used herein, a “heterologous gene” or “heterologous polynucleotide sequence” is defined in relation to the transgenic non-human organism producing such a gene product. A heterologous polypeptide, also referred to as a xenogeneic polypeptide, is defined as a polypeptide having an amino acid sequence or an encoding DNA sequence corresponding to that of a cognate gene found in an organism not consisting of the transgenic non-human animal. Thus, a transgenic mouse harboring a human MC4R gene can be described as harboring a heterologous MC4R gene. A transgene containing various gene segments encoding a heterologous protein sequence may be readily identified, e.g. by hybridization or DNA sequencing, as being from a species of organism other than the transgenic animal. For example, expression of human MC4R amino acid sequences may be detected in the transgenic non-human animals of the invention with antibodies specific for human MC4R epitopes encoded by human MC4R gene segments. A cognate heterologous gene refers to a corresponding gene from another species; thus, if murine MC4R is the reference, human MC4R is a cognate heterologous gene (as is porcine, ovine, or rat MC4R, along with MC4R genes from other species). A mutated endogenous gene sequence can be referred to as a heterologous gene; for example, a transgene encoding a murine MC4R comprising a R165W mutation (which is not known in naturally-occurring murine genomes) is a heterologous transgene with respect to murine and non-murine species.

As used herein, the term “targeting construct” refers to a polynucleotide which comprises: (1) at least one homology region having a sequence that is substantially identical to or substantially complementary to a sequence present in a host cell endogenous gene locus, and (2) a targeting region which becomes integrated into a host cell endogenous gene locus by homologous recombination between a targeting construct homology region and said endogenous gene locus sequence. If the targeting construct is a “hit-and-run” or “in-and-out” type construct (Valancius and Smithies (1991) Mol. Cell. Biol. 11: 1402; Donehower et al. (1992) Nature 356: 215; (1991) J. NIH Res. 3: 59; Hasty et al. (1991) Nature 350; 243, which are incorporated herein by reference), the targeting region is only transiently incorporated into the endogenous gene locus and is eliminated from the host genome by selection. A targeting region may comprise a sequence that is substantially homologous to an endogenous gene sequence and/or may comprise a nonhomologous sequence, such as a selectable marker (e.g., neo, tk, gpt). The term “targeting construct” does not necessarily indicate that the polynucleotide comprises a gene which becomes integrated into the host genome, nor does it necessarily indicate that the polynucleotide comprises a complete structural gene sequence. As used in the art, the term “targeting construct” is synonymous with the terms “targeting vector” and “targeting transgene” as used herein.

The terms “homology region” and “homology clamp” as used herein refer to a segment (i.e., a portion) of a targeting construct having a sequence that substantially corresponds to, or is substantially complementary to, a predetermined endogenous gene sequence, which can include sequences flanking said gene. A homology region is generally at least about 100 nucleotides long, at least about 250 nucleotides long, at least about 500 nucleotides long, or at least about 1000 nucleotides long, or longer. Although there is no demonstrated theoretical minimum length for a homology clamp to mediate homologous recombination, it is believed that homologous recombination efficiency generally increases with the length of the homology clamp. Similarly, the recombination efficiency increases with the degree of sequence homology between a targeting construct homology region and the endogenous target sequence, with optimal recombination efficiency occurring when a homology clamp is isogenic with the endogenous target sequence. The terms “homology clamp” and “homology region” are interchangeable as used herein. Endogenous gene sequences that substantially correspond to, or are substantially complementary to, a transgene homology region are referred to herein as “crossover target sequences” or “endogenous target sequences.”

As used herein, the term “transcriptional unit” or “transcriptional complex” refers to a polynucleotide sequence that comprises a structural gene (exons), a cis-acting linked promoter and other cis-acting sequences necessary for efficient transcription of the structural sequences, distal regulatory elements necessary for appropriate tissue-specific and developmental transcription of the structural sequences, and additional cis sequences important for efficient transcription and translation (e.g., polyadenylation site, mRNA stability controlling sequences).

As used herein, “linked” means in polynucleotide linkage (i.e., phosphodiester linkage). “Unlinked” means not linked to another polynucleotide sequence; hence, two sequences are unlinked if each sequence has a free 5′ terminus and a free 3′ terminus.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

As used herein, the term “correctly targeted construct” refers to a portion of the targeting construct, which is integrated within or adjacent to an endogenous crossover target sequence, such as a portion of an endogenous MC4R gene locus. For example but not limitation, a portion of a targeting transgene encoding neo and flanked by homology regions having substantial identity with endogenous MC4R gene sequences flanking the first exon, is correctly targeted when said transgene portion is integrated into a chromosomal location so as to replace, for example, an exon of the endogenous MC4R gene. In contrast and also for example, if the targeting transgene or a portion thereof is integrated into a nonhomologous region and/or a region not within about 50 kb of an MC4R gene sequence, the resultant product is an incorrectly targeted transgene. It is possible to generate cells having both a correctly targeted transgene(s) and an incorrectly targeted transgene(s). Cells and animals having a correctly targeted transgene(s) and/or an incorrectly targeted transgene(s) may be identified and resolved by PCR and/or Southern blot analysis of genomic DNA.

As used herein, the term “targeting region” refers to a portion of a targeting construct, which becomes integrated into an endogenous chromosomal location following homologous recombination between a homology clamp and an endogenous MC4R gene sequence. Typically, a targeting region is flanked on each side by a homology clamp, such that a double-crossover recombination between each of the homology clamps and their corresponding endogenous MC4R gene sequences results in replacement of the portion of the endogenous MC4R gene locus by the targeting region; in such double-crossover gene replacement targeting constructs the targeting region can be referred to as a “replacement region”. However, some targeting constructs may employ only a single homology clamp (e.g., some “hit-and-run”-type vectors, see, Bradley et al. (1992) Bio/Technology 10: 534, incorporated herein by reference).

As used herein, the term “replacement region” refers to a portion of a targeting construct flanked by homology regions. Upon double-crossover homologous recombination between flanking homology regions and their corresponding endogenous MC4R gene crossover target sequences, the replacement region is integrated into the host cell chromosome between the endogenous crossover target sequences. Replacement regions can be homologous (e.g., have a sequence similar to the endogenous MC4R gene sequence but having a point mutation or missense mutation), nonhomologous (e.g., a neo gene expression cassette), or a combination of homologous and nonhomologous regions. The replacement region can convert the endogenous MC4R allele into an MC4R allele comprising an obesity-causing mutant form of hMC4R, e.g., a R165W mutation; for example, the replacement region can span the portion of the MC4R gene encoding residue 165 of the MC4R polypeptide (or its non-human equivalent) and the replacement region can comprise a sequence encoding R165W. Replacement regions can also include additional sequences, such as epitope tags (e.g., myc tags, fluorescent proteins), as described herein.

The terms “functional disruption” or “functionally disrupted” as used herein means that a gene locus comprises at least one mutation or structural alteration such that the functionally disrupted gene is incapable of directing the efficient expression of functional gene product. For example but not limitation, an endogenous MC4R gene that has a neogene cassette integrated into the exon of a MC4R gene, is not capable of encoding a functional protein that comprises the inactivated exon, and is therefore a functionally disrupted MC4R gene locus. Functional disruption can include the complete substitution of a heterologous MC4R gene locus in place of an endogenous MC4R locus, so that, for example, a targeting transgene that replaces the entire mouse MC4R locus with an obesity-causing mutant form of hMC4R, e.g., a human MC4R R165W mutation allele, which may be functional in the mouse, is said to have functionally disrupted the endogenous murine MC4R locus by displacing it. Preferably, an exon, which is incorporated into the mRNA encoding the MC4R polypeptide is functionally disrupted. Deletion or interruption of essential transcriptional regulatory elements, polyadenylation signal(s), splicing site sequences will also yield a functionally disrupted gene. Functional disruption of an endogenous MC4R gene, may also be produced by other methods (e.g., antisense polynucleotide gene suppression). The term “structurally disrupted” refers to a targeted gene wherein at least one structural (i.e., exon) sequence has been altered by homologous gene targeting (e.g., by insertion, deletion, point mutation(s), and/or rearrangement).

An allele comprising a targeted alteration that interferes with the efficient expression of a functional gene product from the allele is referred to in the art as a “null allele” or “knockout allele”. A “knockout mouse” as used herein refers to a transgenic mouse comprising a null allele or knockout allele, i.e., a transgenic mouse wherein a gene has been rendered inoperative.

As used herein, a “knock-in mouse” refers to a transgenic mouse comprising a gene inserted into a specific locus, i.e., a “targeted” insertion of a gene in the mouse chromosome.

As used herein, the term “pharmacological chaperone” (PC) refers to a cell-permeant, selective ligand of the MC4 receptor, which is able to restore cell surface targeting and function of mis-folded, intracellularly-retained mutant forms of human MC4R (hMC4R). Such mutant forms of MC4R have been associated with obesity. For example, treatment of a cell or animal expressing this type of mutant form of MC4R with a PC may lead to total or partial restoration of cell surface expression and/or signaling activity by the mutant MC4R polypeptide. A PC may be any type of agent such as a chemical compound, a mixture of chemical compounds, a biological macromolecule, or an extract made from biological materials such as bacteria, plants, fungi, or animal (particularly mammalian) cells or tissues. In an embodiment, a PC is a small molecule, low molecular weight chemical compound.

The term “agent” is used herein to denote a chemical compound, a mixture of chemical compounds, a biological macromolecule, or an extract made from biological materials such as bacteria, plants, fungi, or animal (particularly mammalian) cells or tissues.

As used herein, “MC4R polypeptide” refers to a polypeptide that is encoded by the MC4R gene. A mutated MC4R polypeptide having a R165W mutation is a MC4R polypeptide having the arginine at residue 165 replaced with a tryptophan. The wild-type MC4R polypeptide is 332 amino acids long.

As used herein, the terms “obesity-causing mutant” of MC4R, “obesity-causing mutant form” of MC4R, and “mutated hMC4R protein that promotes obesity” are used interchangeably, and refer to a mutated human melanocortin type-4 receptor (hMC4R) protein which is non-functional and is associated with obesity. An obesity-causing mutant of MC4R may, for example, be improperly folded, be retained intracellularly, and/or have impaired signaling activity, as compared to wild-type hMC4R protein. In one embodiment, a mutated hMC4R protein that promotes obesity comprises an arginine at position 165 of the hMC4R protein in place of a tryptophan (R165W mutation).

As used herein, “R165W mutation” refers to a human MC4R polypeptide where the naturally occurring arginine at residue 165 is replaced by a tryptophan, where the N-terminal methionine is residue 1.

In one embodiment, a human MC4R polypeptide comprising the R165W mutation comprises the amino acid sequence set forth in SEQ ID NO: 25 or 27, or a sequence substantially identical thereto, or a variant thereof. In another embodiment, a human MC4R polypeptide comprising the R165W mutation consists of the amino acid sequence set forth in SEQ ID NO: 25 or 27, or a sequence substantially identical thereto, or a variant thereof. In yet another embodiment, a human wild type MC4R polypeptide comprises the amino acid sequence set forth in SEQ ID NO: 26 or 28, or a sequence substantially identical thereto, or a variant thereof. In another embodiment, a human wild type MC4R polypeptide consists of the amino acid sequence set forth in SEQ ID NO: 26 or 28, or a sequence substantially identical thereto, or a variant thereof.

In an embodiment, a human MC4R polypeptide comprising the R165W mutation is encoded by a nucleic acid molecule comprising the sequence set forth in SEQ ID NO: 21, or a sequence substantially identical thereto, or a variant thereof. In another embodiment, a human MC4R polypeptide comprising the R165W mutation is encoded by a nucleic acid molecule consisting of the sequence set forth in SEQ ID NO: 21, or a sequence substantially identical thereto, or a variant thereof. In yet another embodiment, a human wild type MC4R polypeptide is encoded by a nucleic acid molecule comprising the sequence set forth in SEQ ID NO: 22, or a sequence substantially identical thereto, or a variant thereof. In another embodiment, a human wild type MC4R polypeptide is encoded by a nucleic acid molecule consisting of the sequence set forth in SEQ ID NO: 22, or a sequence substantially identical thereto, or a variant thereof.

The phrase “substantially identical” in the context of two nucleic acids or polypeptides, can refer to two or more sequences that have, e.g., at least about 75%, 80%, 85%, 90%, 95%, 98% or 99% or more nucleotide or amino acid residue (sequence) identity, when compared and aligned for maximum correspondence, as measured using any known sequence comparison algorithm, or by visual inspection.

In some embodiments, the invention provides transgenes, targeting constructs, transgenic non-human animals, transgenic non-human cells, and/or transgenic non-human tissues comprising nucleic acids or polypeptides having exemplary sequences of the invention, e.g., SEQ ID NOs: 19, 20, 21, 22, 23, 24, 25, 26, 27, 28, 29, and 30, or sequences substantially identical thereto, e.g., sequences having at least about 75%, 80%, 85%, 90%, 95%, 98% or 99% or more sequence identity thereto. Nucleic acid sequences of the invention can be substantially identical over the entire length of a polypeptide coding region.

Exemplary sequences of the invention are shown in Table 1.

A “substantially identical” amino acid sequence also can include a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from MC4R, resulting in modification of the structure of the polypeptide, without significantly altering its biological activity. For example, amino- or carboxyl-terminal amino acids that are not required for Mc4R biological or receptor activity can be removed. A “substantially identical” nucleotide sequence can also include a sequence that encodes an amino acid sequence that is substantially identical to the amino acid sequence encoded by a reference sequence. In some embodiments, a substantially identical nucleotide sequence can also include a sequence hybridizing under stringent conditions to the complement of a reference sequence.

“Variant” includes polynucleotides or polypeptides of the invention modified at one or more base pairs, codons, introns, exons, or amino acid residues (respectively) yet still retaining the biological activity of a MC4R polypeptide of the invention. The invention also provides transgenes, targeting constructs, transgenic animals, transgenic cells, and/or transgenic tissues comprising sequences in which one or more of the amino acid residues (e.g., of an exemplary polypeptide, e.g., of SEQ ID NOs: 25, 26, 27, or 28) are substituted with a conserved or non-conserved amino acid residue (e.g., a conserved amino acid residue) and such substituted amino acid residue may or may not be one encoded by the genetic code. Conservative substitutions are those that substitute a given amino acid in a polypeptide by another amino acid of like characteristics. Thus, polypeptides of the invention include those with conservative substitutions of sequences of the invention, e.g., the exemplary polypeptides of the invention, including but not limited to the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. Additional variants within the scope of the invention are those in which additional amino acids are fused to the polypeptide, such as a detectable marker protein.

In some aspects, variants, fragments, derivatives and analogs of the exemplary polypeptides of the invention retain the same biological function or activity as the exemplary polypeptides, e.g., MC4R receptor activity, as described herein. In some aspects, variants, fragments, derivatives and analogs of the exemplary nucleic acids of the invention encode polypeptides retaining the same biological function or activity as the exemplary polypeptides.

TABLE 1
Exemplary sequences used in methods, transgenes,
targeting constructs, transgenic non-human animals,
cells, and tissues of the invention.

| DESCRIPTION | SEQUENCE |
|---|---|
| Full DNA sequence of 3HA-hMC4R(R165W)-Venus | SEQ ID NO: 19 |
| construct with neo cassette | |
| Full DNA sequence of myc-hMC4R(WT)-Venus KI | SEQ ID NO: 20 |
| construct with neo cassette | |
| Nucleic acid sequence encoding R165W MC4R | SEQ ID NO: 21 |
| protein | |
| Nucleic acid sequence encoding wild type MC4R | SEQ ID NO: 22 |
| protein | |
| DNA sequence of 3HA-hMC4R(R165W)-Venus con- | SEQ ID NO: 23 |
| struct without neo cassette (after excision of neo | |
| cassette) | |
| DNA sequence of myc-hMC4R(WT)-Venus KI con- | SEQ ID NO: 24 |
| struct without neocassette (after excision of neo | |
| cassette) | |
| R165W MC4R protein | SEQ ID NO: 25 |
| Wild type MC4R protein | SEQ ID NO: 26 |
| Protein sequence of 3HA-hMC4R(R165W)-Venus ex- | SEQ ID NO: 27 |
| pressed in mouse | |
| Protein sequence of myc-hMC4R(WT)-Venus ex- | SEQ ID NO: 28 |
| pressed in mouse | |
| DNA sequence encoding 3HA-hMC4R(R165W)- | SEQ ID NO: 29 |
| Venus protein (encoding SEQ ID NO: 27) | |
| DNA sequence encoding myc-hMC4R(WT)-Venus | SEQ ID NO: 30 |
| protein (encoding SEQ ID NO: 28) | |

Generally, the nomenclature used hereafter and the laboratory procedures in cell culture, molecular genetics, and nucleic acid chemistry and hybridization described below are those well-known and commonly employed in the art. Standard techniques are used for recombinant nucleic acid methods, polynucleotide synthesis, cell culture, and transgene incorporation (e.g., electroporation, microinjection, lipofection). Generally enzymatic reactions, oligonucleotide synthesis, and purification steps are performed according to the manufacturer's specifications. The techniques and procedures are generally performed according to conventional methods in the art and various general references, which are provided throughout this document. The procedures therein are believed to be well-known in the art and are provided for the convenience of the reader. All the information contained therein is incorporated herein by reference.

Chimeric targeted mice are derived according to Hogan, et al., Manipulating the Mouse Embryo: A Laboratory Manual, Cold Spring Harbor Laboratory (1988) and Teratocarcinomas and Embryonic Stem Cells: A Practical Approach, E. J. Robertson, ed., IRL Press, Washington, D.C., (1987).

Embryonic stem cells are manipulated according to published procedures (Teratocarcinomas and Embryonic Stem Cells: A Practical Approach, E. J. Robertson, ed., IRL Press, Washington, D.C. (1987); Zjilstra et al., Nature 342:435-438 (1989); and Schwartzberg et al., Science 246:799-803 (1989)).

In general, the invention encompasses methods and polynucleotide constructs which are employed for generating non-human transgenic animals expressing an MC4R polypeptide comprising an obesity-causing mutant of MC4R, e.g., the R165W mutation. In some embodiments, the non-human transgenic animals expressing an obesity-causing mutant of MC4R, e.g., a R165W hMC4R, also have the endogenous MC4R gene locus functionally disrupted. Non-human transgenic animals expressing a human MC4R polypeptide comprising an obesity-causing mutant of MC4R, e.g., the R165W mutation, and transgenic non-human mammalian cells harboring a transgene encoding an obesity-causing mutant of MC4R, e.g., a human MC4R polypeptide comprising the R165W mutation, are also encompassed, as well as transgenes and targeting constructs used to produce such transgenic cells and animals, transgenes encoding human obesity-causing mutant MC4R polypeptide sequences, e.g., MC4R polypeptide sequences comprising the R165W mutation or other obesity-causing mutants of MC4R, and methods for using the transgenic animals in pharmaceutical screening and as commercial research animals for modeling obesity.

Agents are administered to test animals, such as test mice, which are transgenic and which express an obesity-causing mutant form, e.g., an obesity-causing mutant form, e.g., the R165W mutation, of human MC4R. Particular techniques for producing transgenic mice, which express, e.g., the R165W mutation of MC4R are described hereinafter. It will be appreciated that the preparation of other transgenic animals expressing the human R165W MC4R polypeptide may easily be accomplished, including rats, hamsters, guinea pigs, rabbits, and the like.

The effect of test agents (e.g., potential pharmacological chaperones) on obesity-causing mutants, e.g., R165W, MC4R polypeptide folding, localization at the cell surface, ligand binding, signaling activity and/or MC4R function generally may be measured in test animals in various specimens from the test animals, using art-recognized techniques. Non-limiting examples of such methods are given in the experimental section below. In all cases, it will be necessary to obtain a control value, which is characteristic of the level of MC4R polypeptide folding, localization at the cell surface, or function generally in the test animal in the absence of test compound(s). In cases where the animal is sacrificed, it will be necessary to base such control values on an average or a typical value from other test animals which have been transgenically modified to express the R165W mutation of human MC4R but which have not received the administration of any test compounds or any other substances expected to affect function of human MC4R. Once such control level is determined, test compounds can be administered to additional test animals, where deviation from the average control value indicates that the test compound had an effect on hMC4R function (e.g., reduced degradation, restored or increased proper protein folding, increased localization at the cell surface, increased or restored ligand binding, signaling activity, etc.) in the animal. Test substances which are considered positive, i.e., likely to be beneficial in the treatment of MC4R-induced obesity or other related conditions, will be those which are able to reduce degradation of hMC4R R165W polypeptide; restore, promote, or increase proper hMC4R R165W protein folding; increase localization of hMC4R R165W polypeptide at the cell surface; and/or increase ligand binding or signaling activity by hMC4R R165W polypeptide; preferably by at least 20%, by at least 50%, or by at least 80%.

Test agents can be any molecule, compound, or other substance, which can be added to cell culture or administered to a test animal without substantially interfering with cell or animal viability. Suitable test agents may be small molecules, biological polymers, such as polypeptides, polysaccharides, polynucleotides, and the like. Test compounds will typically be administered to transgenic animals at a dosage of from 1 ng/kg to 10 mg/kg, usually from 10 μg/kg to 1 mg/kg. In an embodiment, a test compound is a candidate pharmacological chaperone. In general pharmacological chaperones are agents which bind selectively to a mutated MC4R inside a cell and restore cell surface expression and/or signaling activity to the mutated MC4R polypeptide. Such agents are attractive therapeutic candidates for treating genetic obesity, e.g., obesity induced by MC4R mutations. It should be understood that any therapeutic candidate for treating obesity may be tested using animals and cells of the invention, regardless of mechanism of action, as appropriate. For example, compounds, which rescue cell surface expression of MC4R via a mechanism such as acting on the quality control system of the cell could be tested using animals and cells of the invention. Test compounds, which are able to have a positive effect on hMC4R function as described above are considered likely to be beneficial in the treatment of MC4R-induced obesity, other MC4R-related conditions, and/or obesity in general.

The present invention further comprises pharmaceutical compositions incorporating a compound selected by the above-described methods and including a pharmaceutically acceptable carrier. Such pharmaceutical compositions should contain a therapeutic or prophylactic amount of at least one compound identified by the methods of the present invention. The pharmaceutically acceptable carrier can be any compatible, non-toxic substance suitable to deliver the compounds to an intended host. Sterile water, alcohol, fats, waxes, and inert solids may be used as the carrier. Pharmaceutically acceptable adjuvants, buffering agents, dispersing agents, and the like may also be incorporated into pharmaceutical compositions. Preparation of pharmaceutical conditions incorporating active agents is well described in the medical and scientific literature. See, for example, Remington's Pharmaceutical Sciences, Mack Publishing Company, Easton, Pa., 16th Ed., 1982, the disclosure of which is incorporated herein by reference.

Pharmaceutical compositions provided herein are suitable for systemic administration to the host, including both parenteral, topical, and oral administration. Pharmaceutical compositions may be administered parenterally, i.e. subcutaneously, intramuscularly, or intravenously. Thus, the present invention provides compositions for administration to a subject, where the compositions comprise a pharmaceutically acceptable solution of the identified compound in an acceptable carrier, as described above. In an embodiment, the subject is a human. In another embodiment, the subject is an obese human. In a particular embodiment, the subject is a human having a mutation in the MC4R gene or a human suffering from MC4R-induced obesity. The MC4R-induced obesity may be early-onset obesity.

In an embodiment of the invention, a transgene encoding a heterologous MC4R protein comprising the R165W mutation is transferred into a fertilized embryo or an ES cell to produce a transgenic non-human animal that expresses a human MC4R polypeptide comprising the R165W mutation. A transgene encoding a heterologous R165W mutation MC4R protein comprises structural sequences encoding a heterologous R165W mutation MC4R protein, and generally also comprises linked regulatory elements that drive expression of the heterologous R165W mutation MC4R protein in the non-human host. However, endogenous regulatory elements in the genome of the non-human host may be exploited by integrating the transgene sequences into a chromosomal location containing functional endogenous regulatory elements which are suitable for the expression of the heterologous structural sequences. Such targeted integration is usually performed by homologous gene targeting as described supra, wherein the heterologous transgene would comprise at least one homology clamp.

When a heterologous transgene relies on its own regulatory elements, suitable transcription elements and polyadenylation sequence(s) are included. At least one promoter is linked upstream of the first structural sequence in an orientation to drive transcription of the heterologous structural sequences. Sometimes the promoter from the naturally-occurring heterologous gene is used (e.g., a human MC4R promoter is used to drive expression of a human R165W mutation MC4R transgene). Alternatively, the promoter from the endogenous cognate MC4R gene may be used (e.g., the murine MC4R promoter is used to drive expression of a human R165W mutation MC4R transgene). Alternatively, a transcriptional regulatory element heterogenous with respect to both the transgene encoding sequences and the non-human host animal can be used (e.g., a rat promoter and/or enhancer operably linked to a nucleotide sequence encoding human R165W mutation MC4R, wherein the transgene is introduced into mice).

In some embodiments, it is preferable that the transgene sequences encoding the R165W mutation MC4R polypeptide are under the transcriptional control of promoters and/or enhancers (and/or silencers) which are not operably linked in naturally-occurring MC4R genes (i.e., non-MC4R promoters and/or enhancers). For example, some embodiments will employ transcriptional regulatory sequences which confer high level expression and/or in a cell type-specific expression pattern (e.g., a neuron-specific promoter, sim-1 gene promoter, BDNF promoter, etc.). Various promoters having different strengths may be substituted in the discretion of the practitioner, however it is essential that the promoter function in the non-human host and it is desirable in some embodiments that the promoter drive expression in a developmental pattern or cell type-specific pattern (and at expression levels) similar to a naturally-occurring MC4R gene in a parallel host animal lacking the transgene.

A heterologous transgene generally encodes a full-length MC4R polypeptide (e.g., 332 amino acids). The heterologous transgene may comprise a polynucleotide spanning the entire genomic MC4R gene or a portion thereof, may comprise a single contiguous coding segment (e.g., cDNA), or may comprise a combination thereof. Frequently, the transgene encodes a human MC4R polypeptide sequence comprising the R165W mutation, however transgenes encoding non-human MC4R polypeptides comprising the R165W mutation may also be used.

In some embodiments, transgenes encoding MC4R polypeptide sequences with mutations causing improper protein folding or intracellular retention of the receptor, where the mutation is other than the R165W mutation, are used.

Transgenes encoding MC4R mutated polypeptides will frequently also comprise one or more linked selectable markers (infra). For example, fluorescent tags may be linked to the MC4R polypeptides in the transgenes to allow detection of the protein encoded by the transgene, e.g., by detecting fluorescence. In an embodiment, a fluorescent tag is green fluorescent protein (GFP), blue fluorescent protein, red fluorescent protein, cyan fluorescent protein, or yellow fluorescent protein (e.g., encoded by the Venus sequence). Other tags, of which many are known in the art, may also be linked to the MC4R polypeptides in the transgenes. For example, human influenza hemagglutinin (HA) tags may be linked to the MC4R polypeptides in the transgenes. Typically, 1, 2, 3, 4, 5 or 6 HA tags are linked. In an embodiment, 3 HA tags are linked to the MC4R polypeptides in the transgenes.

In some embodiments, transgenes comprise more than one marker or tag. For example, a transgene may be double-tagged, i.e., comprise more than one marker or tag. Markers or tags can be used, for example, to detect a transgene in situ, using methods such as immunohistochemistry or direct fluorescence. In one embodiment, a transgene comprises a MC4R coding region linked at one end (3′ or 5′) to a gene encoding a fluorescent protein, such as yellow fluorescent protein, and at the other end to a gene encoding a detectable tag, such as HA. In embodiments, where more than one marker or tag is present, a transgene can be detected in situ by double-labelling, e.g., by both immunohistochemistry and direct fluorescence. It is noted that, in embodiments where more than one marker or tag is present, e.g., two tags are present, the presence of the two tags can allow easy detection of the receptor and also quantification of the fraction of receptor that is expressed at the cell surface, e.g., by dual FACS.

Such markers or tags may also be flanked by regions allowing excision of the marker or tag after insertion into the mouse chromosome. For example, flippase recognition target (FRT) sites which allow site-directed recombination by the flippase recombinase (FLP) may flank a gene encoding a marker or tag. Other site-specific recombinase systems are known in the art and may be used in targeting vectors, such as, but not limited to, Lox (e.g., LoxP) sequences which are recombined by the Cre recombinase.

Transgenes encoding heterologous MC4R polypeptides comprising the R165W mutation molecules may be transferred into the non-human host genome in several ways. A heterologous transgene may be targeted to a specific predetermined chromosomal location by homologous targeting, as described supra for gene targeting. Heterologous transgenes may be transferred into a host genome in pieces, by sequential homologous targeting, to reconstitute a complete heterologous gene in an endogenous host chromosomal location. In contradistinction, a heterologous transgene may be randomly integrated separately from or without using a MC4R gene targeting construct. A heterologous transgene may be co-transferred with an MC4R gene targeting construct and, if desired, selected for with a separate, distinguishable selectable marker and/or screened with PCR or Southern blot analysis of selected cells. Alternatively, a heterologous transgene may be introduced into ES cells prior to or subsequent to introduction of a MC4R gene targeting construct and selection therefor. A heterologous transgene may be introduced into the germline of a non-human animal by nonhomologous transgene integration via pronuclear injection, and resultant transgenic lines bred into a homozygous knockout background having functionally disrupted cognate endogenous MC4R gene. Homozygous knockout mice can also be bred and the heterologous R165W mutation MC4R transgene introduced into embryos of knockout mice directly by standard pronuclear injection or other means known in the art.

In some embodiments, endogenous non-human MC4R alleles are functionally disrupted so that expression of endogenously encoded MC4R is suppressed or eliminated, so as to not interfere or contaminate transgene-encoded MC4R comprising the R165W mutation. In one variation, an endogenous MC4R allele is converted to comprise the R165W mutation by homologous gene targeting.

Gene targeting, which is a method of using homologous recombination to modify a mammalian genome, can be used to introduce changes into cultured cells. By targeting a gene of interest in embryonic stem (ES) cells, these changes can be introduced into the germlines of laboratory animals to study the effects of the modifications on whole organisms, among other uses. The gene targeting procedure is accomplished by introducing into tissue culture cells a DNA targeting construct that has a segment homologous to a target locus and which also comprises an intended sequence modification (e.g., insertion, deletion, point mutation). The treated cells are then screened for accurate targeting to identify and isolate those which have been properly targeted. A common scheme to disrupt gene function by gene targeting in ES cells is to construct a targeting construct, which is designed to undergo a homologous recombination with its chromosomal counterpart in the ES cell genome. The targeting constructs are typically arranged so that they insert additional sequences, such as a positive selection marker, into coding elements of the target gene, thereby functionally disrupting it. Targeting constructs usually are insertion-type or replacement-type constructs (Hasty et al. (1991) Mol. Cell. Biol. 11: 4509).

The invention encompasses methods to produce non-human animals (e.g., non-primate mammals, e.g., rodents, e.g., mice) that have the endogenous MC4R gene inactivated by gene targeting with a homologous recombination targeting construct. Typically, a non-human MC4R gene sequence is used as a basis for producing PCR primers that flank a region that will be used as a homology clamp in a targeting construct. The PCR primers are then used to amplify, by high fidelity PCR amplification (Mattila et al. (1991) Nucleic Acids Res. 19: 4967; Eckert, K. A. and Kunkel, T. A. (1991) PCR Methods and Applications 1: 17; U.S. Pat. No. 4,683,202, which are incorporated herein by reference), a genomic sequence from a genomic clone library or from a preparation of genomic DNA, preferably from the strain of non-human animal that is to be targeted with the targeting construct. The amplified DNA is then used as a homology clamp and/or targeting region. Thus, homology clamps for targeting a non-human MC4R gene may be readily produced on the basis of nucleotide sequence information available in the art and/or by routine cloning. General principles regarding the construction of targeting constructs and selection methods are reviewed in Bradley et al. (1992) Bio/Technology 10: 534.

Endogenous non-human MC4R genes may be functionally disrupted and, optionally, may be replaced by transgenes encoding MC4R comprising the R165W mutation.

Targeting constructs can be transferred into pluripotent stem cells, such as murine embryonal stem cells, wherein the targeting constructs homologously recombine with a portion of an endogenous MC4R gene locus and create mutation(s) (i.e., insertions, deletions, rearrangements, sequence replacements, and/or point mutations) which prevent the functional expression of the endogenous MC4R gene.

In an embodiment, targeting constructs of the invention are employed to replace a portion of an endogenous MC4R gene with an exogenous sequence (i.e., a portion of a targeting transgene); for example, the exon of a MC4R gene may be replaced with a substantially identical portion that contains a mutation, e.g., a mutation which disrupts proper folding of the polypeptide or causes intracellular retention of the polypeptide, e.g., a R165W mutation.

In another embodiment, an endogenous MC4R gene in a non-human host is functionally disrupted by homologous integration of a cognate heterologous MC4R gene comprising the R165W mutation, such that the cognate heterologous MC4R gene substantially replaces the endogenous MC4R gene, and preferably completely replaces the coding sequences of the endogenous MC4R gene. Preferably, the heterologous R165W mutation MC4R gene is linked, as a consequence of homologous integration, to regulatory sequences (e.g., an enhancer) of the endogenous MC4R gene so that the heterologous R165W mutation gene is expressed under the transcriptional control of regulatory elements from the endogenous MC4R gene locus. Non-human hosts which are homozygous for such replacement alleles (i.e., a host chromosomal MC4R locus which encodes a cognate heterologous R165W mutation MC4R gene product) may be produced according to methods described herein. Such homozygous non-human hosts generally will express a heterologous R165W mutation MC4R protein but do not express the endogenous MC4R protein. Most usually, the expression pattern of the heterologous R165W mutation MC4R gene will substantially mimic the expression pattern of the endogenous MC4R gene in the naturally-occurring (non-transgenic) non-human host. For example but not limitation, a transgenic mouse having human R165W mutation MC4R gene sequences replacing the endogenous murine MC4R gene sequences and which are transcriptionally controlled by endogenous murine regulatory sequences generally will be expressed similarly to the murine MC4R in naturally occurring non-transgenic mice.

Generally, a replacement-type targeting construct is employed for homologous gene replacement. Double-crossover homologous recombination between endogenous MC4R gene sequences and homology clamps flanking the replacement region (i.e., the heterologous R165W mutation MC4R encoding region) of the targeting construct result in targeted integration of the heterologous R165W mutation MC4R gene segments. Usually, the homology clamps of the transgene comprise sequences which flank the endogenous MC4R gene segments, so that homologous recombination results in concomitant deletion of the endogenous MC4R gene segments and homologous integration of the heterologous gene segments. Substantially an entire endogenous MC4R gene may be replaced with a heterologous MC4R gene comprising the R165W mutation by a single targeting event. One or more selectable markers, usually in the form of positive or negative selection expression cassettes, may be positioned in the targeting construct replacement region.

ES cells harboring a heterologous R165W mutation MC4R gene, such as a replacement allele, may be selected in several ways. First, a selectable marker (e.g., neo, gpt, tk) may be linked to the heterologous R165W mutation MC4R gene (e.g., in an intron or flanking sequence) in the targeting construct so that cells having a replacement allele may be selected for. Most usually, a heterologous MC4R gene targeting construct will comprise both a positive selection expression cassette and a negative selection expression cassette, so that homologously targeted cells can be selected for with a positive-negative selection scheme (Mansour et al. (1988) op. cit., incorporated herein by reference).

In some embodiments, transgenes and/or targeting constructs of the invention comprise a nucleic acid molecule having the sequence set forth in SEQ ID NO: 19 (for R165W mutation MC4R) or SEQ ID NO: 20 (for wild type MC4R), or a sequence substantially identical thereto, or a variant thereof. In other embodiments, transgenes and/or targeting constructs of the invention consist of sequences set forth in SEQ ID NO: 19 (for R165W mutation MC4R) or SEQ ID NO: 20 (for wild type MC4R) or a sequence substantially identical thereto, or a variant thereof. In further embodiments, transgenes and/or targeting constructs of the invention comprise a nucleic acid molecule having the sequence set forth in SEQ ID NO: 21 (for R165W mutation MC4R) or SEQ ID NO: 22 (for wild type MC4R) or a sequence substantially identical thereto, or a variant thereof. In other embodiments, transgenes and/or targeting constructs of the invention comprise a nucleic acid molecule having the sequence set forth in SEQ ID NO: 23 or 29 (for R165W mutation MC4R) or SEQ ID NO: 24 or 30 (for wild type MC4R) or a sequence substantially identical thereto, or a variant thereof).

In some embodiments, transgenes and/or targeting constructs of the invention comprise a nucleic acid molecule encoding MC4R polypeptide comprising the R165W mutation, wherein the nucleic acid molecule has the sequence set forth in SEQ ID NO: 21 or a sequence substantially identical thereto, or a variant thereof. In other embodiments, transgenes and/or targeting constructs of the invention comprise a nucleic acid molecule encoding wild type polypeptide, wherein the nucleic acid molecule has the sequence set forth in SEQ ID NO: 22 or a sequence substantially identical thereto, or a variant thereof.

In some embodiments, transgenes and/or targeting constructs of the invention comprise a nucleic acid molecule encoding MC4R polypeptide comprising the R165W mutation, wherein the MC4R polypeptide comprising the R165W mutation has the amino acid sequence set forth in SEQ ID NO: 25 or 27 or a sequence substantially identical thereto, or a variant thereof. In other embodiments, transgenes and/or targeting constructs of the invention comprise a nucleic acid molecule encoding wild type MC4R polypeptide, wherein the wild type MC4R polypeptide, has the amino acid sequence set forth in SEQ ID NO: 26 or 28 or a sequence substantially identical thereto, or a variant thereof.

### Targeting Constructs

Several gene targeting techniques have been described, including but not limited to: co-electroporation, “hit-and-run”, single-crossover integration, and double-crossover recombination (Bradley et al. (1992) Bio/Technology 10: 534). The invention can be practiced using essentially any applicable homologous gene targeting strategy known in the art. The configuration of a targeting construct depends upon the specific targeting technique chosen. For example, a targeting construct for single-crossover integration or “hit-and-run” targeting need only have a single homology clamp linked to the targeting region, whereas a double-crossover replacement-type targeting construct requires two homology clamps, one flanking each side of the replacement region.

Targeting constructs of the invention comprise at least one MC4R homology clamp linked in polynucleotide linkage (i.e., by phosphodiester bonds) to a targeting region. A homology clamp has a sequence, which substantially corresponds to, or is substantially complementary to, an endogenous MC4R gene sequence of a non-human host animal, and may comprise sequences flanking the MC4R gene.

Although no lower or upper size boundaries for recombinogenic homology clamps for gene targeting have been conclusively determined in the art, targeting constructs are generally at least about 50 to about 100 nucleotides long, or at least about 250 to about 500 nucleotides long, or at least about 1000 to about 2000 nucleotides long, or longer. Construct homology regions (homology clamps) are generally at least about 50 to about 100 bases long, or at least about 100 to about 500 bases long, or at least about 750 to about 2000 bases long. In an embodiment, homology regions of about 7 to about 8 kilobases in length are preferred, with one embodiment having a first homology region of about 7 kilobases flanking one side of a replacement region and a second homology region of about 1 kilobase flanking the other side of said replacement region. The length of homology (i.e., substantial identity) for a homology region may be selected at the discretion of the practitioner on the basis of the sequence composition and complexity of the endogenous MC4R gene target sequence(s) and guidance provided in the art (Hasty et al. (1991) Mol. Cell. Biol. 11: 5586; Shulman et al. (1990) Mol. Cell. Biol. 10: 4466).

Targeting constructs have at least one homology region having a sequence that substantially corresponds to, or is substantially complementary to, an endogenous MC4R gene sequence (e.g., an exon sequence, an enhancer, a promoter, an intronic sequence, or a flanking sequence within about 3-20 kb of a MC4R gene). Such a targeting transgene homology region serves as a template for homologous pairing and recombination with substantially identical endogenous MC4R gene sequence(s). In targeting constructs, such homology regions typically flank the replacement region, which is a region of the targeting construct that is to undergo replacement with the targeted endogenous MC4R gene sequence (Berinstein et al. (1992) Mol. Cell. Biol. 12: 360). Thus, a segment of the targeting construct flanked by homology regions can replace a segment of an endogenous MC4R gene sequence by double-crossover homologous recombination. Homology regions and targeting regions are linked together in conventional linear polynucleotide linkage (5′ to 3′ phosphodiester backbone). Targeting constructs are generally double-stranded DNA molecules, most usually linear.

Without wishing to be bound by any particular theory of homologous recombination or gene conversion, it is believed that in such a double-crossover replacement recombination, a first homologous recombination (e.g., strand exchange, strand pairing, strand scission, strand ligation) between a first targeting construct homology region and a first endogenous MC4R gene sequence is accompanied by a second homologous recombination between a second targeting construct homology region and a second endogenous MC4R gene sequence, thereby resulting in the portion of the targeting construct that was located between the two homology regions replacing the portion of the endogenous MC4R gene that was located between the first and second endogenous MC4R gene sequences. For this reason, homology regions are generally used in the same orientation (i.e., the upstream direction is the same for each homology region of a transgene to avoid rearrangements). Double-crossover replacement recombination thus can be used to delete a portion of an endogenous MC4R gene and concomitantly transfer a nonhomologous portion (e.g., a neogene expression cassette) into the corresponding chromosomal location. Double-crossover recombination can also be used to add a nonhomologous portion into an endogenous MC4R gene without deleting endogenous chromosomal portions. Upstream and/or downstream from the nonhomologous portion may be a gene which provides for identification of whether a double-crossover homologous recombination has occurred; such a gene may be, e.g., the HSV tk gene which can be used for negative selection.

A positive selection expression cassette encodes a selectable marker which affords a means for selecting cells which have integrated targeting transgene sequences spanning the positive selection expression cassette. A negative selection expression cassette encodes a selectable marker which affords a means for selecting cells which do not have an integrated copy of the negative selection expression cassette. Thus, by a combination positive-negative selection protocol, it is possible to select cells that have undergone homologous replacement recombination and incorporated the portion of the transgene between the homology regions (i.e., the replacement region) into a chromosomal location by selecting for the presence of the positive marker and for the absence of the negative marker.

In an embodiment, expression cassettes for inclusion in targeting constructs of the invention encode and express a selectable drug resistance marker and/or a HSV thymidine kinase enzyme. Suitable drug resistance genes include, for example: gpt (xanthine-guanine phosphoribosyltransferase), which can be selected for with mycophenolic acid; neo (neomycin phosphotransferase), which can be selected for with G418 or hygromycin; and DFHR (dihydrofolate reductase), which can be selected for with methotrexate (Mulligan and Berg (1981) Proc. Natl. Acad. Sci. (U.S.A.) 78: 2072; Southern and Berg (1982) J. Mol. Appl. Genet. 1: 327; which are incorporated herein by reference). In an embodiment, a neomycin gene is linked to the MC4R polypeptides in transgenes of the invention.

In an embodiment, an epitope tag is linked to MC4R polypeptides in transgenes of the invention. For example, 3xHA, a myc tag or a fluorescent protein may be linked to the N-terminus or C-terminus of a MC4R polypeptide in a transgene. In an embodiment, one or more epitope tags, e.g., two epitope tags, are linked to MC4R polypeptides in transgenes of the invention. In an embodiment, 3xHA is linked to the N-terminus of an obesity-causing mutant of hMC4R. In an embodiment, myc is linked to the N-terminus of a wild-type hMC4R polypeptide (WT-hMC4R) in a transgene. In an embodiment, a fluorescent protein, e.g., yellow fluorescent protein, is linked to the C-terminus of a hMC4R polypeptide. Such epitope tags can allow immunodetection of the protein encoded by the transgene. Presence of a fluorescence protein, e.g., yellow fluorescent protein, allows detection of the protein by direct fluorescence. Presence of two tags can allow easy detection of the receptor protein as well as quantification of the fraction of receptor that is expressed at the cell surface, e.g., by dual FACS.

Selection for correctly targeted recombinants will generally employ at least positive selection, wherein a nonhomologous expression cassette encodes and expresses a functional protein (e.g., neo or gpt) that confers a selectable phenotype to targeted cells harboring the endogenously integrated expression cassette, so that, by addition of a selection agent (e.g., G418 or mycophenolic acid) such targeted cells have a growth or survival advantage over cells which do not have an integrated expression cassette.

In some embodiments, selection for correctly targeted homologous recombinants also employs negative selection, so that cells bearing only nonhomologous integration of the transgene are selected against. Typically, such negative selection employs an expression cassette encoding the herpes simplex virus thymidine kinase gene (HSV tk) positioned in the transgene so that it should integrate only by nonhomologous recombination. Such positioning generally is accomplished by linking the HSV tk expression cassette (or other negative selection cassette) distal to the recombinogenic homology regions so that double-crossover replacement recombination of the homology regions transfers the positive selection expression cassette to a chromosomal location but does not transfer the HSV tk gene (or other negative selection cassette) to a chromosomal location. A nucleoside analog, gancyclovir, which is preferentially toxic to cells expressing HSV tk, can be used as the negative selection agent, as it selects for cells which do not have an integrated HSV tk expression cassette. FIAU may also be used as a selective agent to select for cells lacking HSV tk. In order to reduce the background of cells having incorrectly integrated targeting construct sequences, a combination positive-negative selection scheme may be used (Mansour et al. (1988) op. cit., incorporated herein by reference).

Generally, targeting constructs of the invention preferably include: (1) a positive selection expression cassette flanked by two homology regions that are substantially identical to host cell endogenous MC4R gene sequences, and (2) a distal negative selection expression cassette. However, targeting constructs, which include only a positive selection expression cassette can also be used. Typically, a targeting construct will contain a positive selection expression cassette which includes a neogene linked downstream (i.e., towards the carboxy-terminus of the encoded polypeptide in translational reading frame orientation) of a promoter such as the HSV tk promoter or the pgk promoter. More typically, the targeting transgene will also contain a negative selection expression cassette which includes an HSV tk gene linked downstream of a HSV tk promoter.

In some embodiments, targeting constructs of the invention have homology regions that are highly homologous to the predetermined target endogenous DNA sequence(s), preferably isogenic (i.e., identical sequence). Isogenic or nearly isogenic sequences may be obtained by genomic cloning or high-fidelity PCR amplification of genomic DNA from the strain of non-human animals, which are the source of the ES cells used in the gene targeting procedure.

Vectors containing a targeting construct are typically grown in E. coli and then isolated using standard molecular biology methods, or may be synthesized as oligonucleotides. Direct targeted inactivation which does not require prokaryotic or eukaryotic vectors may also be done. Targeting transgenes can be transferred to host cells by any suitable technique, including microinjection, electroporation, lipofection, biolistics, calcium phosphate precipitation, and viral-based vectors, among others. Other methods used to transform mammalian cells include the use of Polybrene, protoplast fusion, and others (See, generally, Sambrook et al. Molecular Cloning: A Laboratory Manual, 2d ed., 1989, Cold Spring Harbor Laboratory Press, Cold Spring Harbor, N.Y., which is incorporated herein by reference).

For making transgenic non-human animals (which include homologously targeted non-human animals), embryonal stem cells (ES cells) are generally used. Murine ES cells, such as AB-1 line grown on mitotically inactive SNL76/7 cell feeder layers (McMahon and Bradley (1990) Cell 62: 1073) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 27-45), and the CCE line (Robertson et al. (1986) Nature 323: 445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal). The blastocysts containing the injected ES cells are allowed to develop in the uteri of pseudopregnant non-human females and are born as chimeric mice. The resultant transgenic mice are chimeric for cells having inactivated endogenous MC4R loci and are backcrossed and screened for the presence of the correctly targeted transgene(s) by PCR or Southern blot analysis on tail biopsy DNA of offspring so as to identify transgenic mice heterozygous for the inactivated MC4R locus. By performing the appropriate crosses, it is possible to produce a transgenic non-human animal homozygous for functionally disrupted MC4R alleles, and optionally also harboring a transgene encoding a heterologous MC4R polypeptide comprising the R165W mutation. Such transgenic animals are substantially incapable of making an endogenous MC4R gene product but express the R165W mutation heterologous MC4R.

### Commercial Research and Screening Uses

Non-human animals comprising transgenes which encode R165W mutation MC4R can be used commercially to screen for agents having the effect of restoring cell surface expression or function (e.g., signaling) of the mutated MC4R polypeptide. Such agents can be developed as pharmaceuticals for treating MC4R deficiency, obesity, or other related conditions. Transgenic animals of the present invention exhibit severe obesity and other symptoms of MC4R-deficiency, and can be used for pharmaceutical screening and as disease models for obesity and other MC4R-related conditions. Transgenic animals of the present invention thus have many uses, including but not limited to: identifying compounds that effect or affect MC4R protein folding, cell surface expression, ligand binding or signaling; testing candidate therapeutic agents for obesity or other MC4R-related conditions, e.g., to obtain proof-of-concept in an animal model; and providing disease models for investigating MC4R-related pathological conditions (e.g., early-onset obesity and the like, as well as processes other than energy homeostasis, such as bone metabolism or inflammation). Such transgenic animals can be commercially marketed to researchers, among other uses.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1

### Generation of Knock-in Mouse Model

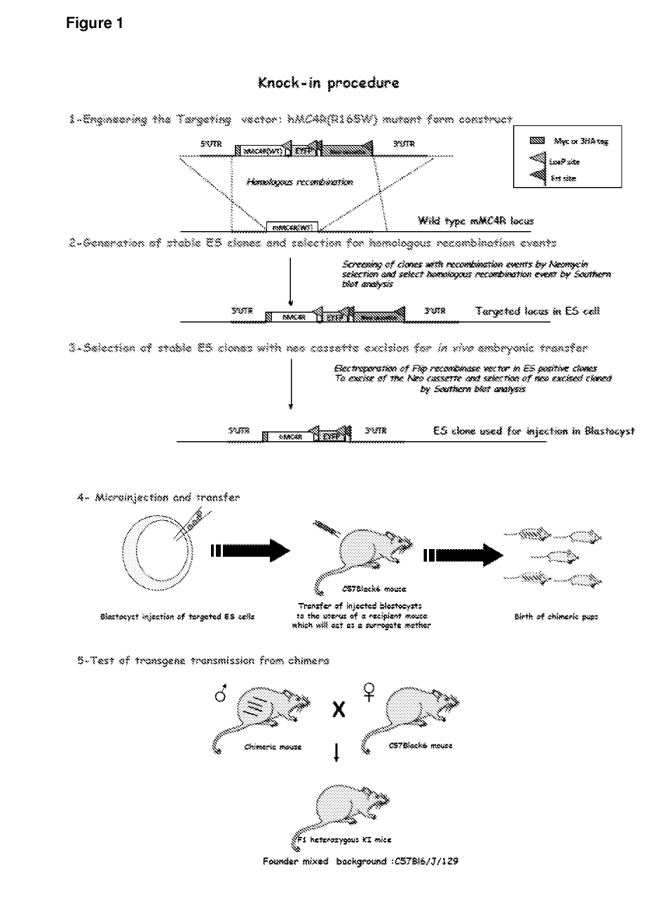

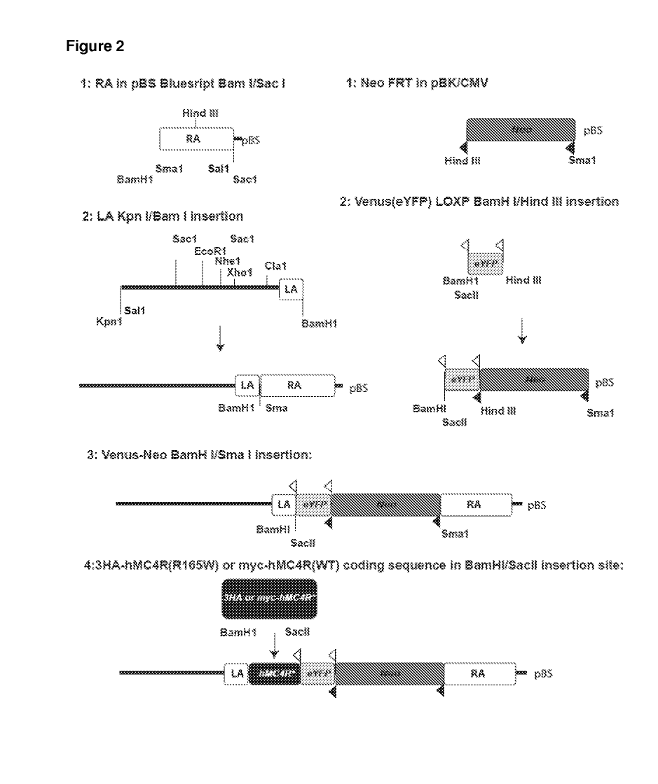

A knock-in mouse model carrying either a double tagged obesity-causing mutant form of human melanocortin 4 receptor (hMC4R) or a double-tagged wild-type form of hMC4R at the mouse MC4R locus was generated. For the knock-in procedure, the following steps were followed: i) a targeting vector was engineered; ii) stable ES clones were generated and selected for homologous recombination events; iii) stable ES clones were selected with neo cassette excision for in vivo embryonic transfer; iv) microinjection and transfer was performed; v) transgene transmission from chimera was tested, to determine whether colonization of the germ line occurred; vi) male founders for generation of a mouse line were selected; and vii) a lineage on mixed background was started. A schematic diagram illustrating these steps is shown in FIG. 1.

The “knock-in” mouse lines generated here express either an obesity-causing mutant form of the human MC4R (hMC4R) or a wild-type form of hMC4R in the receptor's mouse locus, thus replacing the mouse gene and creating humanized MC4R mice. The mutation R165W was selected because of its prevalence in humans and because of its capacity to be efficiently restored functionally by pharmacological chaperones in cellular systems (Rene, P. et al., 2010, J. Pharmacol. Exp. Ther., 335:520-532).

i. Engineering of the Targeting Construct.

The general strategy to make the targeting construct was based on the MC4R endogenous gene structure and sequence to define restriction enzymes to use for building the construct and for Southern blot screening strategy. Design of the targeting construct was based on independent PCR amplification of each piece of the construct and distinct restriction digest and ligation cycles to build the complete targeting vector (see FIG. 2).

Amplification of the Left homologous Arm (LA)-2.7 kb and the Right homologous Arm (RA)-1.5 Kb was done using PCR, from mouse genomic DNA with the same background as the ES cells used for embryonic transfer.

A positive selective neomycin cassette was flanked by FRT sites to be able to subsequently remove the cassette using Flip recombinase.

Human melanocortin 4 receptor (hMC4R) coding sequence was modified by:

Inserting a mutation in ApaI site located in the coding sequence to be able to distinguish by restriction digest the transgenic allele (hMC4R) from the endogenous one (mMC4R);
Inserting a mutation replacing the Arginine (R) at position 165 in the coding sequence of hMC4R by a Tryptophan (W) for the mutant form of hMC4R;
Fusing in the same open reading frame as the MC4R gene coding sequence a 3xHA Tag or a myc sequence containing the initiation site at the 5′ end of the coding sequence of hMC4R; and
Fusing in the same open reading frame as the MC4R gene coding sequence a yellow fluorescent protein coding sequence (Venus) at the 3′ end of the coding sequence of hMC4R. This yellow fluorescent protein coding sequence (Venus) was flanked by loxP sites in order to be able to subsequently remove the Venus coding sequence using CRE recombinase.

All key pieces of the transgenic construct generated by PCR were sequenced to confirm that no mutations were introduced during amplification. We also tested the use of Cre and Flip recombinases and sequenced products of the recombination to make sure that the excision was correct and in frame.

We then linearized the final product (referred to herein as “targeting construct”) with the SalI restriction enzyme before introducing the targeting construct into ES cells by electroporation.

ii. Generating Stable ES Clones and Selecting for Homologous Recombination Events.

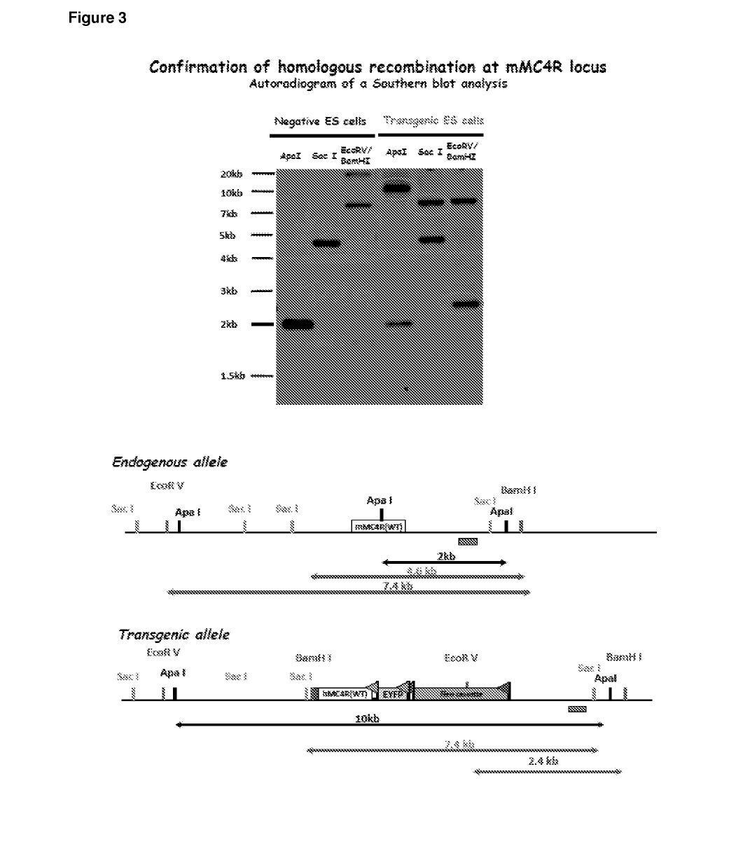

A total of 170 ES clones selected in presence of neomycin were screened by Southern blot hybridization analysis of ApaI-digested-genomic DNA with the flanking probe shown in FIG. 3 (red thick bar). One clone showed the predicted 10 kb targeted ApaI DNA fragment in addition to the expected 2 kb wild-type fragment. In order to further confirm homologous recombination at the mMC4R locus, this clone was further analyzed by Southern blot hybridization with the flanking probe shown in FIG. 3 (red thick bar) after ApaI, SacI, EcoRV/BamHI restriction digestion of ES positive and negative clones. The restriction pattern obtained for the positive clone was as expected for a targeted insertion at the mMC4R locus (data shown in FIG. 3).

iii. Screening of ES Clones Containing the Transgenic Allele without the Selective Neomycin Cassette at the mMC4R Locus.

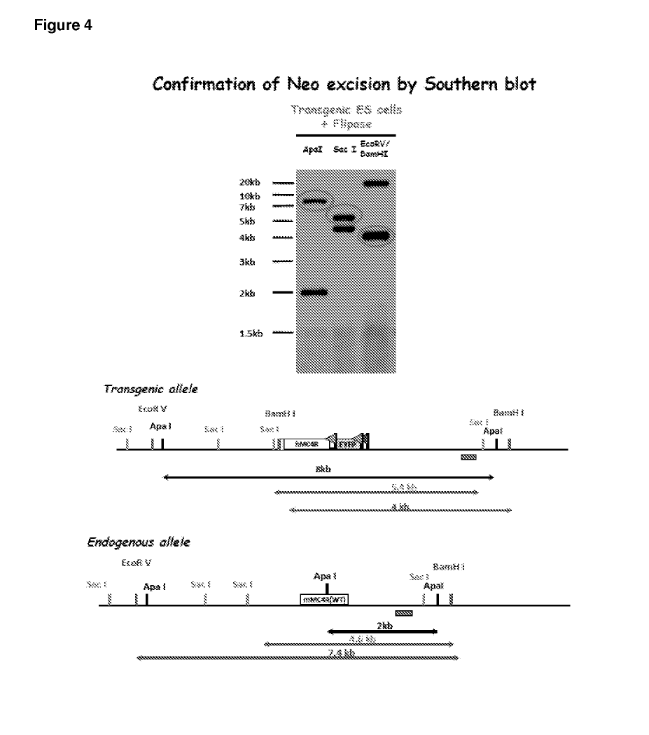

Electroporation of the plasmid coding for Flip recombinase was done to excise the neomycin cassette prior to transfer of ES positive cells to blastocysts. A total of 68 ES clones were screened by Southern blot hybridization analysis of SacI-digested genomic DNA with the flanking probe shown in FIG. 4 (red thick bar). Four ES clones with the predicted pattern showed a 5.4 kb targeted SacI DNA fragment in addition to the expected 7.4 kb wild-type fragment. Further restriction digests of positive clones were done to confirm excision of the neomycin cassette after ApaI, SacI, EcoRV/BamHI restriction digestion by Southern blot hybridization with the flanking probe shown in FIG. 4 (red thick bar) (data shown in FIG. 4).

iv. Screening for Transgenic Mice from Transmission Test with Chimeric Mice Carrying the Transgenic Allele.

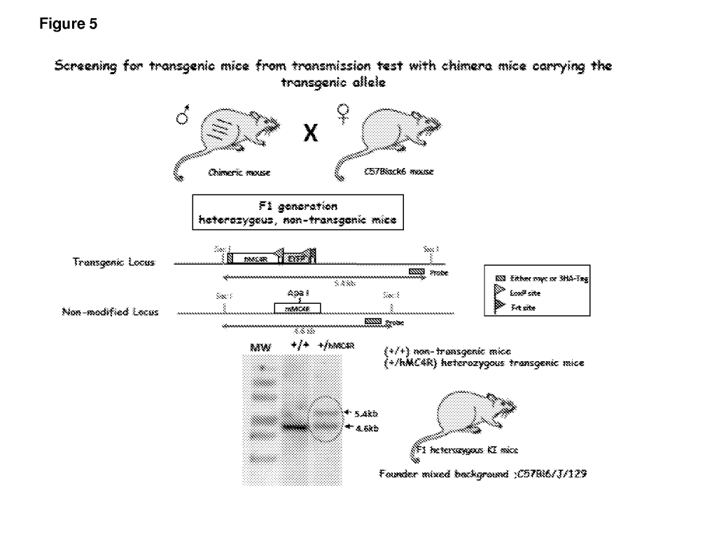

The targeted ES positive clones with the neomycin cassette excised were injected into C57Bl/6J blastocysts to generate chimeras. Male chimeras were bred with C57Bl/6J females and germ line transmission in offspring was determined by the presence of the targeted hMC4R (R165W) or (WT) allele by Southern blot hybridization of SacI digested tail DNA with the flanking probe, as shown in FIG. 5 (red thick bar). Mice carrying the transgenic allele with the predicted pattern showing a 5.4 kb targeted SacI DNA fragment in addition to the expected 7.4 kb wild-type fragment were selected as founders.

v. Breeding of the R165W or WT-hMC4R Knock-in (KI) Mouse Line to Obtain Each Genotype for Phenotype Characterization.

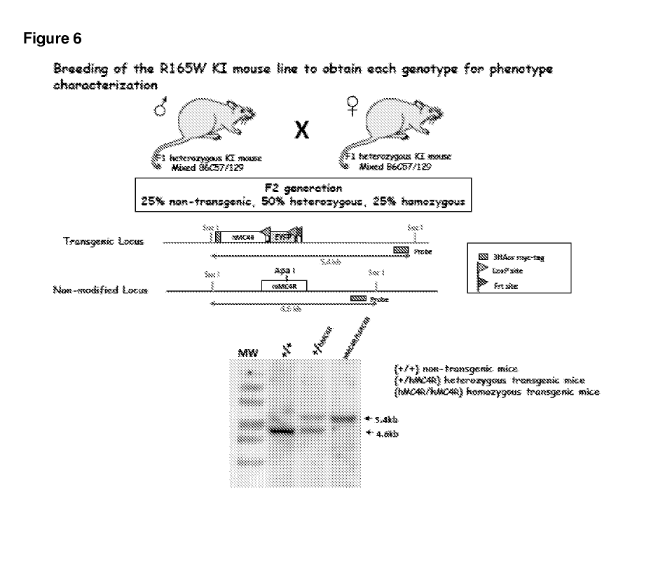

Offspring heterozygous for the mutation from founder breeding were then bred together and were genotyped by Southern blot hybridization of SacI digested tail DNA with the flanking probe (red thick bar) as shown in FIG. 6. Mice carrying the mutant allele with the predicted pattern showing a 5.4 kb targeted SacI DNA fragment in addition to the expected 7.4 kb wild-type fragment were heterozygous for the mutation. Mice with the 5.4 kb targeted SacI DNA fragment were homozygous for the mutation.

### Example 2

### Phenotypic Characterization of Knock-in Mouse Model Carrying Human Mutant Form of MC4R

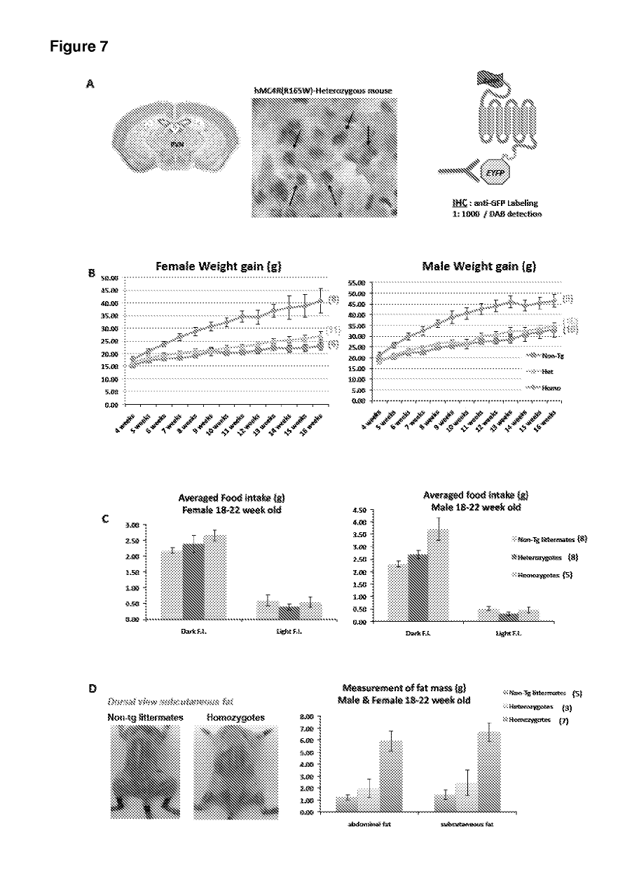

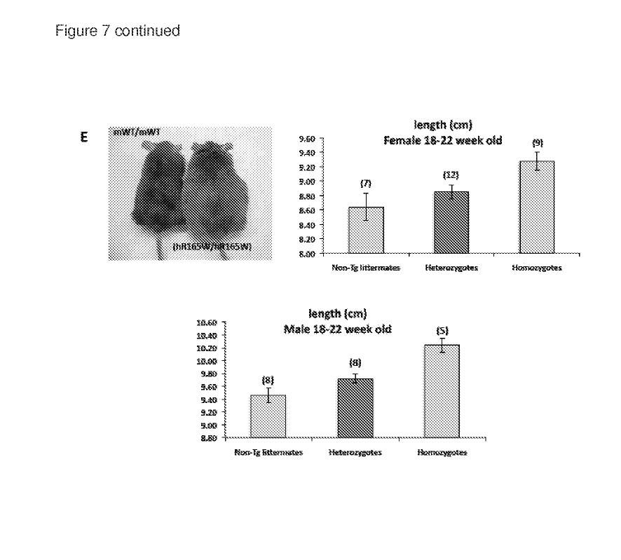

To determine hMC4R(R165W) brain expression in our Knock-in mouse model, immunohistochemistry was performed on frozen brain slices from heterozygous knock-in mice using anti-GFP antibody and DAB labeling. GFP immunoreactivity was detected in neurons located in the paraventricular nucleus known to express MC4R, confirming expression of the mutant allele (FIG. 7A).

F2 animals were maintained on a chow diet ad libitum and their weight was monitored regularly. The weight of MC4R-knock-in mice and their wild-type littermates was largely indistinguishable for the first 4 weeks. However, by approximately 5 weeks of age, most of the homozygous mutants, both male and female, were heavier than their wild-type siblings of the same sex, and by 7 weeks of age all of the homozygous hMC4R (R165W) mutant mice were heavier than controls (FIG. 7B). By 15 weeks of age, homozygous mutant females were on average twice as heavy as their wild-type siblings, while homozygous mutant males were approximately 1.3 fold heavier than wild-type controls.

To determine whether food consumption was increased in hMC4R(R165W) homozygous mice, basal food intake on regular chow diet was measured in individualized mice of 18-22 weeks of age during dark and light cycles. We observed during the nocturnal phase an increase of 12% in food consumption in females homozygous for hMC4R(R165W) and a 17% increase in males homozygous for hMC4R(R165W), compared to littermate controls (FIG. 7C). We also observed an increase in abdominal and subcutaneous fat mass of about 6-fold, compared to non-transgenic littermates (FIG. 7D).

Alterations in linear growth have been reported in MC4R deficient mice and humans. In order to determine whether our knock-in mouse model exhibited the same phenotype, body length (snout-anus) measurements of F2 progeny were taken at 18-22 weeks of age. As shown in FIG. 7E, hMC4R(R165W) homozygous mice were longer than wild-type littermates (mean length was increased approximately 11% relative to wild-type littermates for both genders).

These results show that the knock-in mouse model displays the same phenotype (hyperphagia, onset of weight gain at 5-week-old, increase in fat mass and linear growth) as null MC4R mouse models. These results also confirm that the mice recapitulate phenotypic features of MC4R-deficient humans. These mice represent the first model of MC4R-related obesity and the first model for a GPCR conformational disease.

Phenotypic characterization was made on mice backcrossed once into the C57Bl/6J genetic background.

### Example 3

### Phenotypic Characterization of a Knock-in Mouse Model Carrying Human Wild-Type Form of MC4R

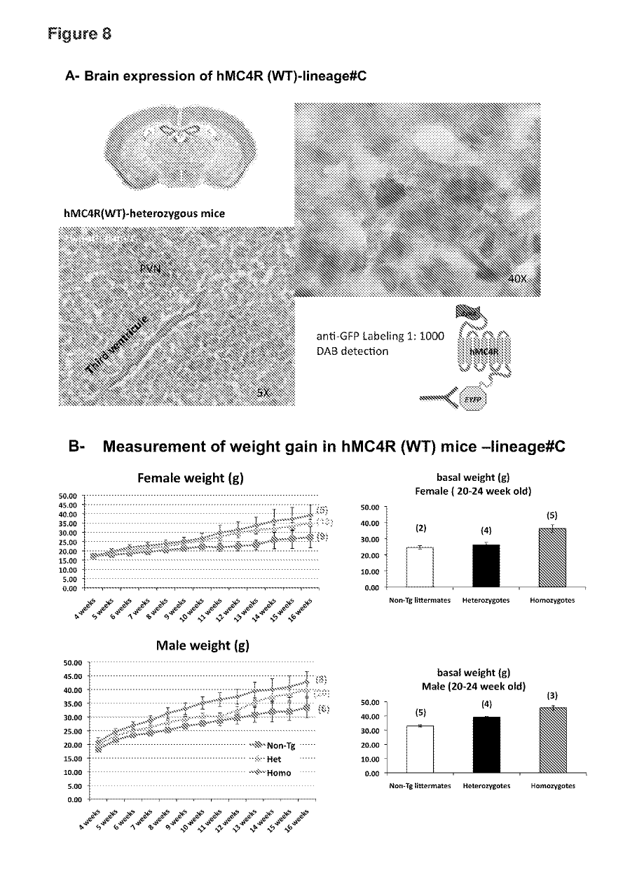

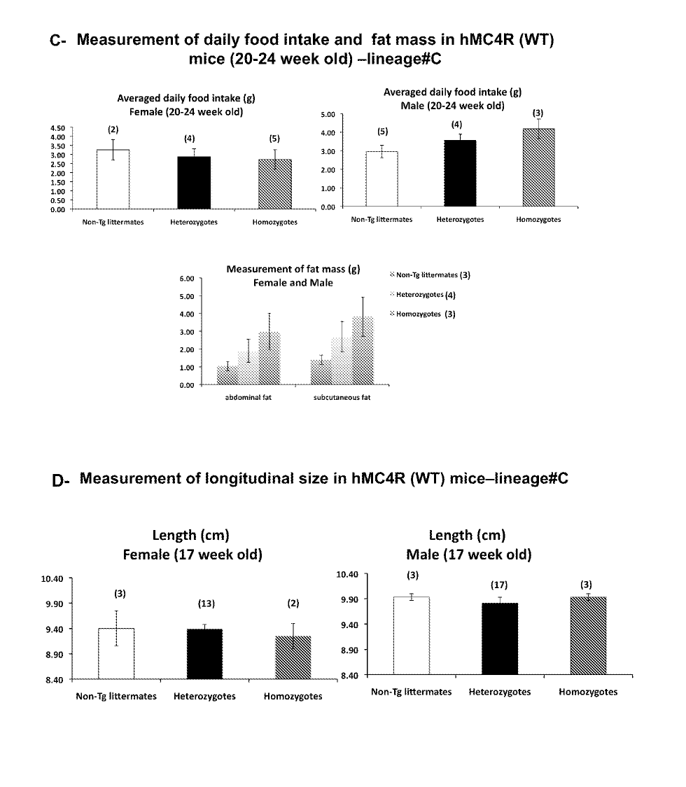

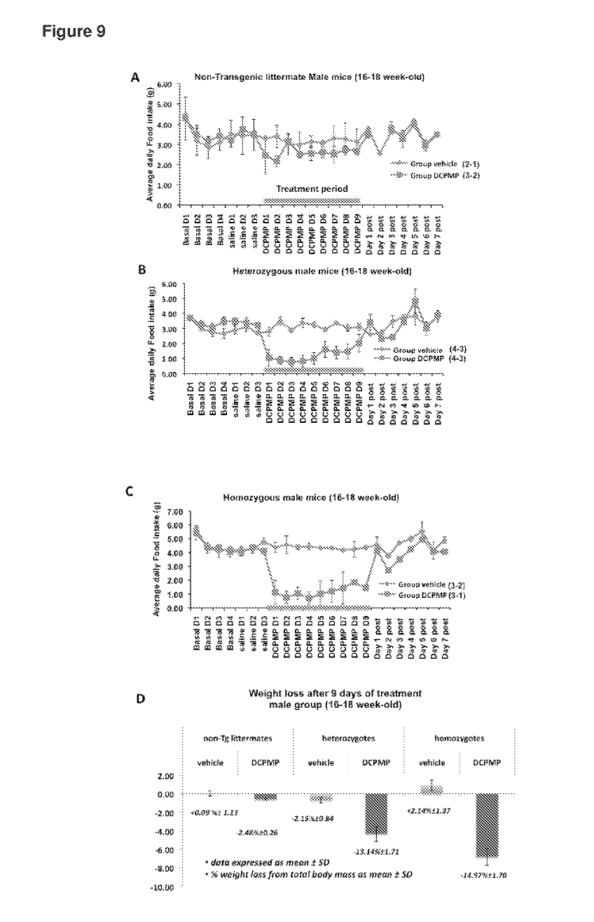

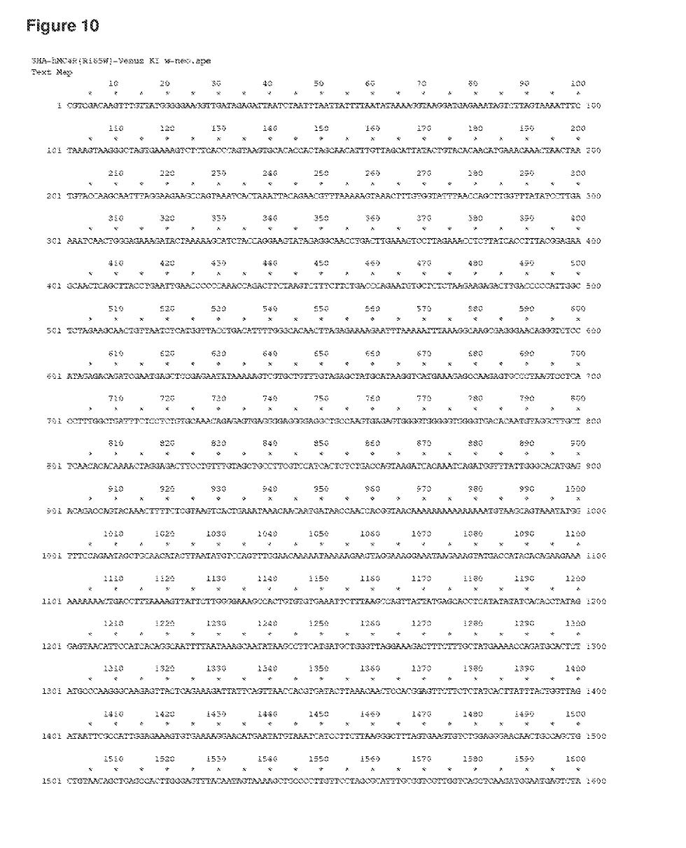

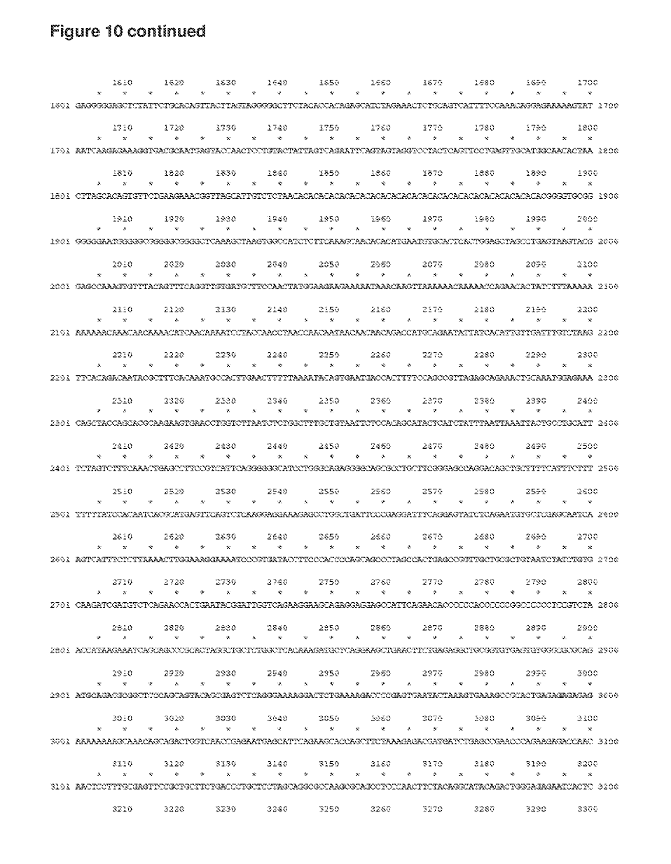

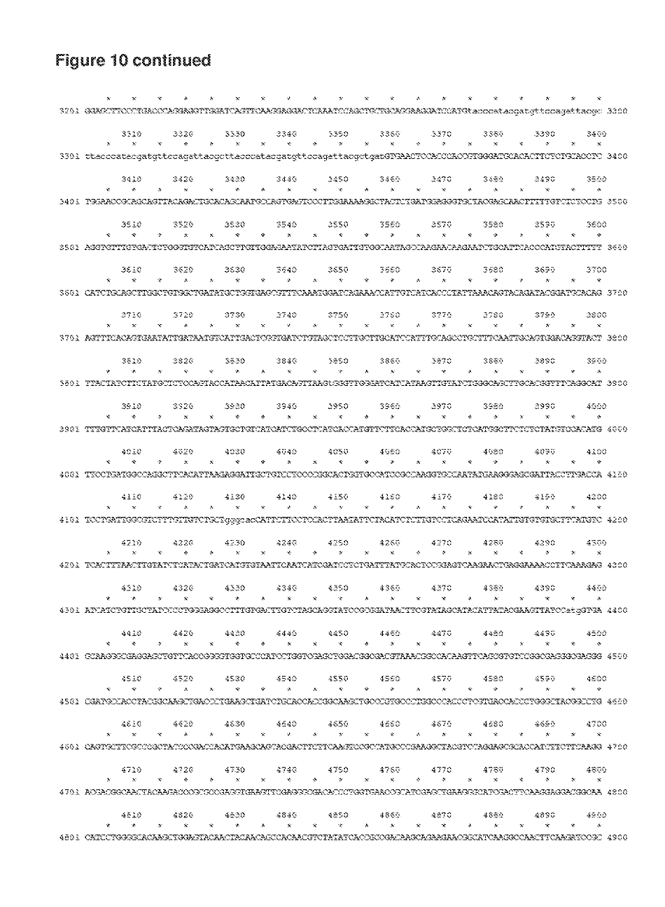

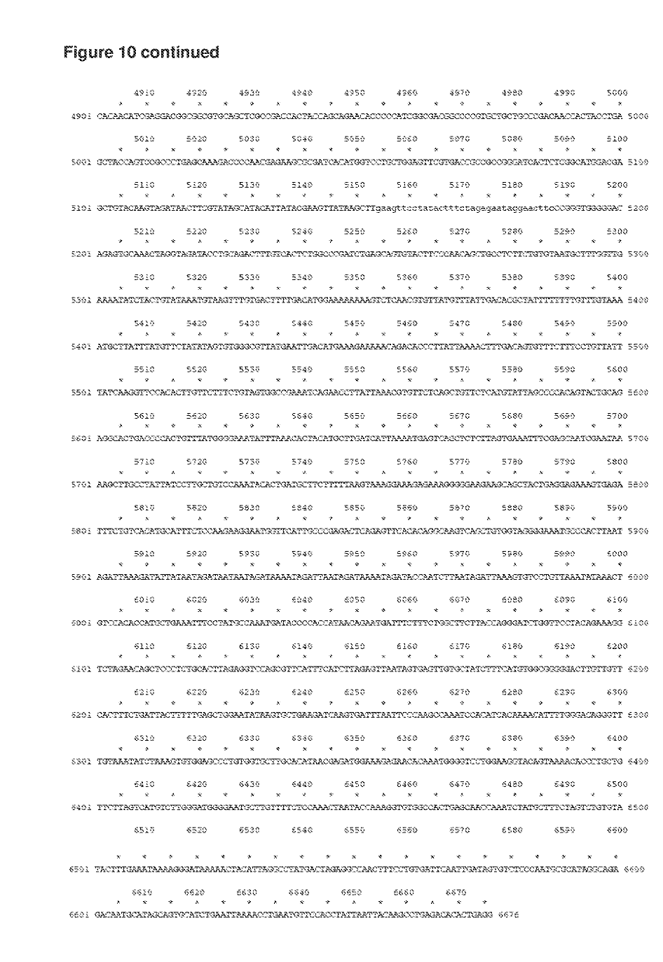

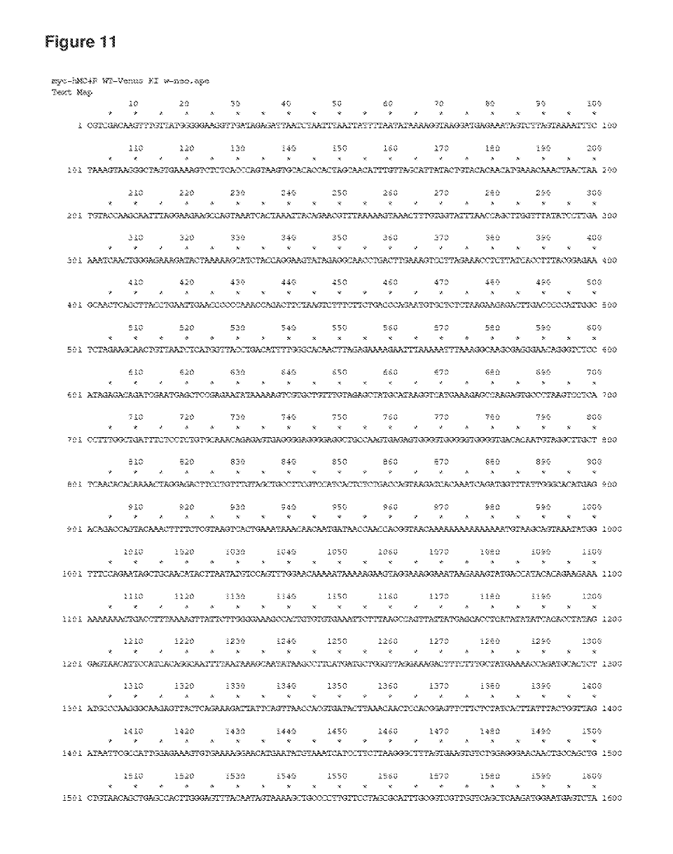

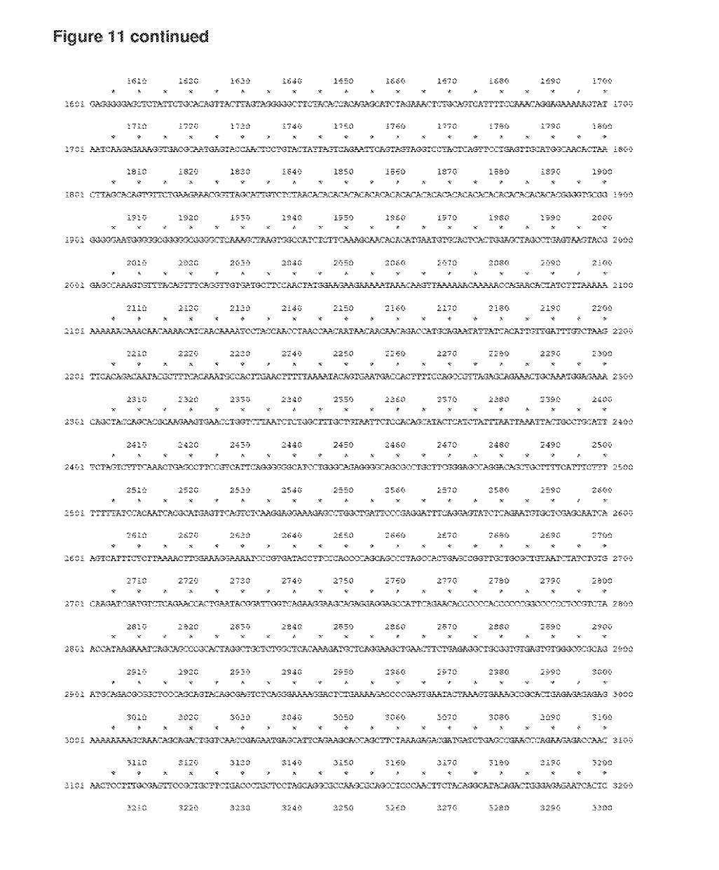

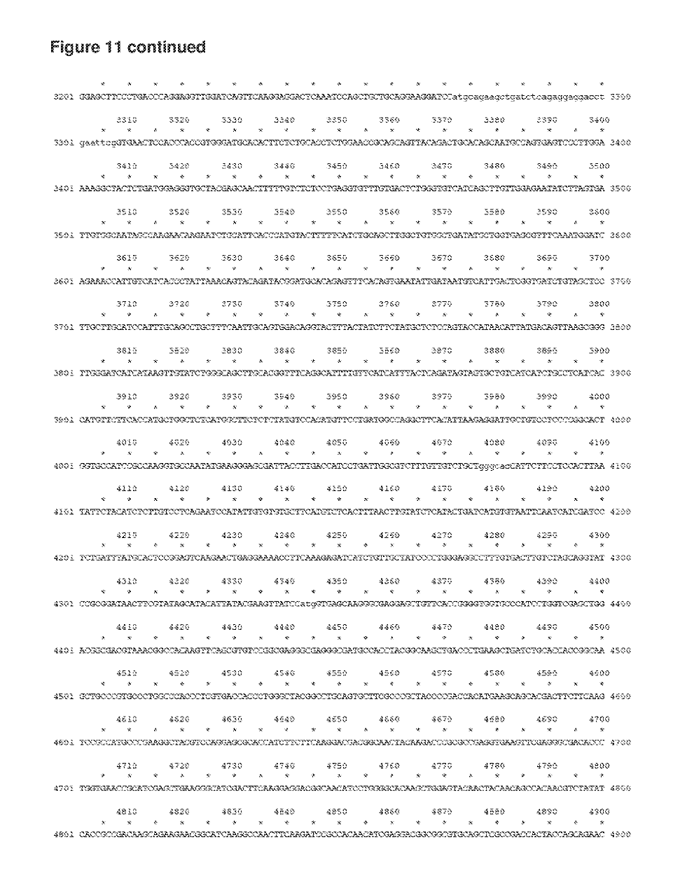

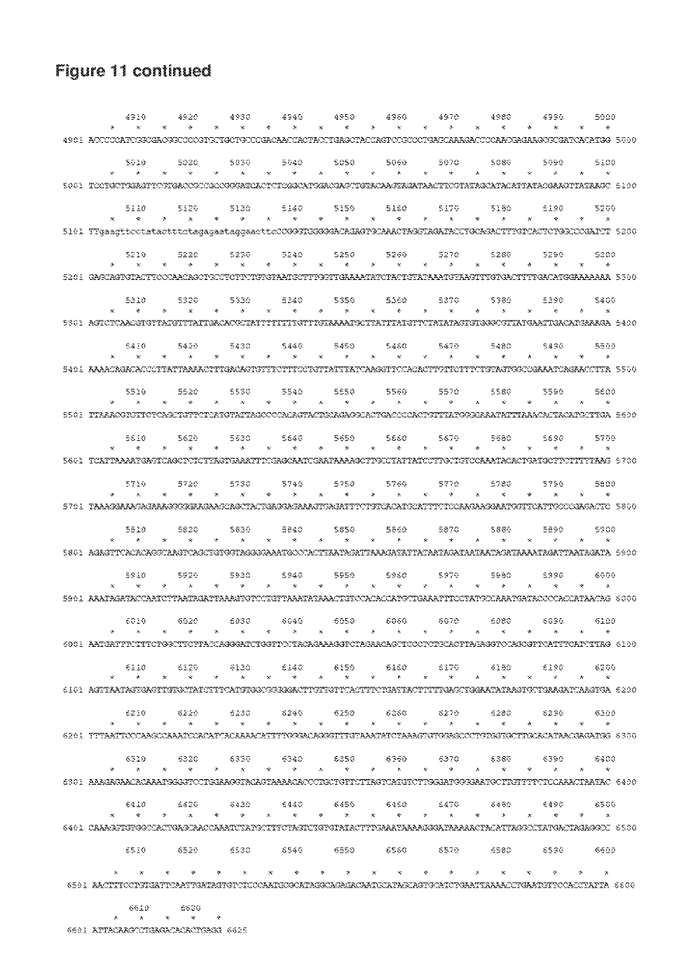

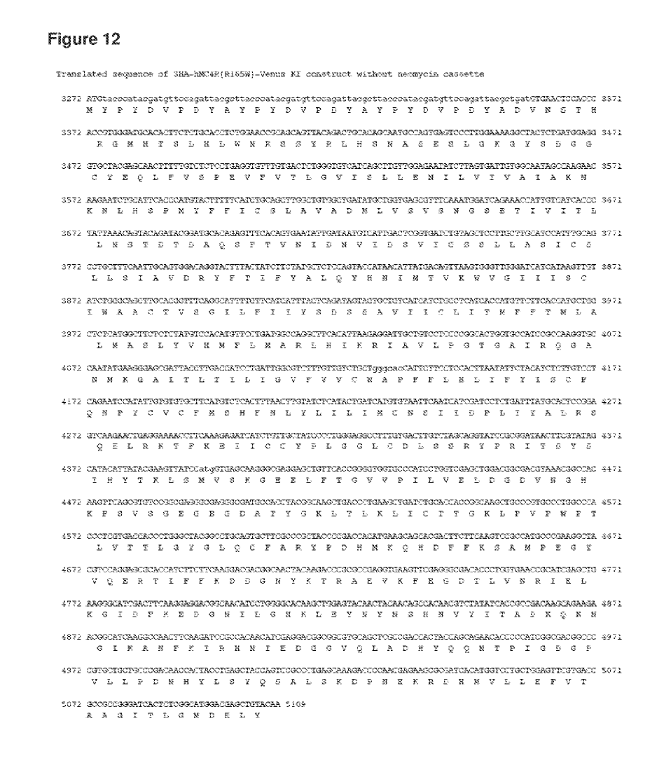

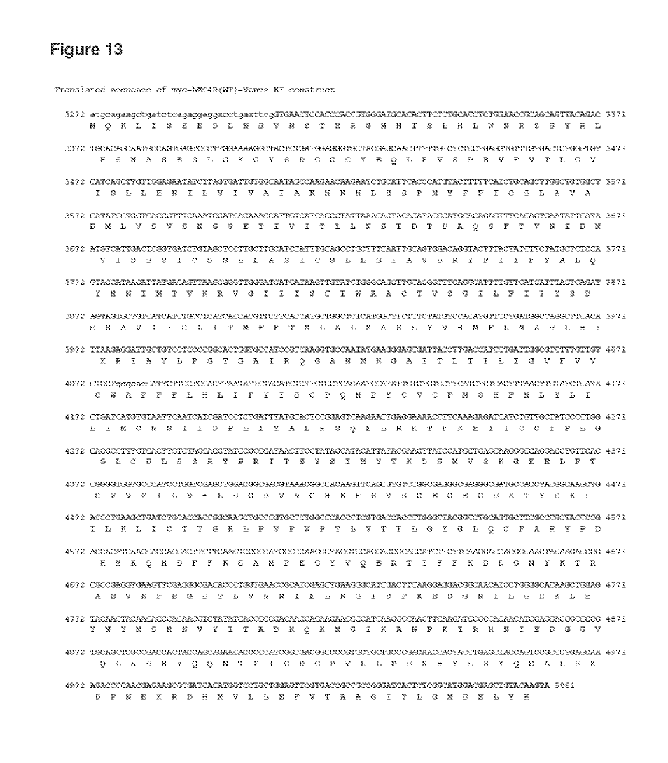

In addition to transgenic mice bearing the hMC4R mutant gene (hMC4R(R165W)), knock-in transgenic mice wherein the murine MC4R gene was replaced by the wild-type human MC4R gene (hMC4R(WT)) flanked by a myc tag at the N-terminus and a YFP venus protein at the C-terminus were also generated, using standard procedures such as those described herein. Expression of the wild-type human MC4R transgene (hMC4R(WT)) on frozen brain slices from heterozygous knock-in mice was assessed by immunohistochemistry using anti-GFP antibody and DAB labeling (FIG. 8A). Animals homozygous for the hMC4R(WT) transgene developed excess weight with age and increased fat mass, without modifying significantly their food intake. This increase in weight was about 2-fold less than that observed in mice homozygous for hMC4R(R165W). Moreover, these mice did not show any increase in longitudinal size (snout-anus length)(FIG. 8D).

Phenotypic characterization was made on mice backcrossed once into the C57Bl/6J genetic background.

### Example 4

### In Vivo Test of DCPMP Compound on Knock-in Mouse Model Carrying Human Mutant Form of MC4R

Mice were intraperitoneally injected one dose daily with vehicle or N-((2R)-3(2,4-dichlorophenyl)-1-(4-(2-((1-methoxypropan-2-ylamino)methyl)phenyl)piperazin-1-yl)-1-oxopropan-2-yl)propionamide (DCPMP) at 30 mg per kg one hour before light off, for 9 days. Results of food intake and weight loss measurements are shown in FIG. 8. In addition to reducing food intake, DCPMP treatment was also able to promote significant weight loss, up to 13% and 15% of total weight in initially overweight heterozygous and homozygous mice, respectively. Intriguingly, food consumption was also slightly reduced at the beginning of DCPMP treatment, in non-transgenic littermate mice. In the course of in vitro studies with MC4R selective pharmacological chaperones (PCs), we found that treatment of cells with PCs not only restored cell surface expression and function of mutant MC4Rs but also increased cell surface targeting of the wild type (WT) receptor, resulting in increased signaling activity in response to agonist stimulation. This result is consistent with the fact that efficacy of folding for many membrane proteins, including GPCRs, is not 100%. In a study characterizing biosynthesis of a delta-opioid receptor, we found that as little as 50% of the synthesized receptor reached the folding state required to escape the endoplasmic-reticulum quality control system and reach the cell surface (Petäjä-Repo, U. E. et al., J. Biol. Chem., 275:13727-36 (2000)) The finding that PCs also increase the number of functional MC4Rs at the cell surface suggests that general obesity not resulting from MC4R mutations could also be treated with MC4R-selective PCs.

### Materials and Methods

### Mutagenesis and Tag Replacement

The mutant form of hMC4R (R165W) N-terminally tagged with 3xHA and containing an APAI site in the coding sequence was generated by site-directed mutagenesis using overlap extension (Ho, S. N. et al., Gene, 77: 51-59 (1989)). This procedure involved two steps: 1) introduction of the desired base substitution into the hMC4R(WT) receptor cDNA using specifically designed complementary and overlapping primers, followed by 2) amplification of the mutated cDNA using the polymerase chain reaction (PCR). Each point mutation was inserted by PCR performed with Phusion taq polymerase (Fynnzymes, NEB, Ontario, Canada) using specific primers containing the mutation complementary to opposite strands of the hMC4R (WT) template (*) and either a T7-Forward primer (5′-ATTAATACGACTCACTATAGGG-3′)(SEQ ID NO: 1) or a pcDNA3.1-Reverse primer (5′-AGAACGTGGACTCCAACGTCAAAG-3′)(SEQ ID NO: 2). *R165W Forward primer was 5′-G ACA GTT AAG TGG GTT GGG ATC ATC-3′ (SEQ ID NO: 3).

The first fragment was generated using the T7-Forward primer and the reverse/antisense primer complementary to forward sequence above. The second fragment was generated using the pcDNA3.1-Reverse primer and the forward/sense primer (sequence above). The WT form of hMC4R N-terminally tagged with myc and containing an APAI site in the coding sequence was generated by PCR using the myc-BAMHI-BGLII Forward primer and the pcDNA3.1-Reverse primer. The myc-BAMHI-BGLII Forward primer has the following sequence: 5′-TCGGATCCCCGAGATCTCACCATGGCATCAATGCAGAAGCTGATCTCAGAGGA GGACCTGAATTCGGTGAACTCCACCCACCGT-3′ (SEQ ID NO: 4).

The 3xHA-hMC4R (WT) cDNA (Missouri S&T cDNA Resource center, USA) served as template in the PCR reaction to generate both the mutant form for 3HA-hMC4R (R165W) and the myc tagged WT form for myc-hMC4R(WT). Reaction conditions were 30 cycles of 94° C. (30 s), 55° C. (1 min), and 72° C. (1 min). The fragments were then purified using the QIAGEN PCR purification kit (QIAGEN Mississauga, ON, Canada) and combined in the overlap extension reaction using T7-Forward and pcDNA3.1-Reverse primers described. Full length mutant PCR products were purified with QIAGEN gel extraction kit (QIAGEN Mississauga, ON, Canada) and inserted after restriction digest in KpnI/XhoI pcDNA3.1(+) vector. All PCR products were sequenced to confirm the presence of the desired mutation and absence of unwanted mutations.

Construction of 3HA-hMC4R(R165W) and the Myc-hMC4R(WT) Modified to be Compatible with the Targeting Vector

Plasmids described in Example 1 were used as template to generate a 3HA-hMC4R(R165W) or a myc-hMC4R(WT) coding sequence containing a mutated APA I site integrated in targeting vector. BamHI and SacII restriction sites were inserted to be compatible with the targeting backbone vector. The following primers were used:

3HA-BAMHI Forward:
(SEQ ID NO: 5)
5′-TAA GCT TGG ATC CAT GTA CCC ATA CGA TGT TC -3′;
myc-BAMHI Forward:
(SEQ ID NO: 6)
5′- CCATGGGATCCATGCAGAAGCTGATCTCAGAGG-3′;
*APAI Forward:
(SEQ ID NO: 7)
5′-GTT GTC TGC TGG GCA CCA TTC TTC CTC CAC-3′;
and
3′-hMC4R SAC II Reverse:
(SEQ ID NO: 8)
5′-CCT CCC CGC GGA TAC CTG CTAGAC AAG TCA CAA AGG
CCT CCC -3′.

The first fragment was generated using the primers 3HA-BAMHI Forward or myc-BAMHI Forward and the reverse/antisense primer complementary to the forward sequence above (APA I primer). The second fragment was generated using the 3′-hMC4R Sac II-Reverse primer and the forward/sense primer (APAI sequence above). A 3xHA-hMC4R (R165W) cDNA (described above) served as template in a PCR reaction to generate a construct to be inserted in a targeting vector. Reaction conditions were 25 cycles of 94° C. (30 s), 67° C. (1 min), and 72° C. (1 min). Fragments were then purified using the QIAGEN PCR purification kit (QIAGEN Mississauga, ON, Canada) and combined in an overlap extension reaction using 3HA-BAMHI-Forward or myc-BAMHI and 3′-hMC4R SacII-Reverse primers. Full-length PCR products were purified with QIAGEN gel extraction kit (QIAGEN Mississauga, ON, Canada) and digested with BamHI and SacII. All PCR products were sequenced to confirm the presence of desired modifications and absence of unwanted mutations.

### Construction of the Targeting Vector

The right arm (RA) (DNA sequence from ENSMUSG00000047259) was amplified from genomic DNA extracted from an ES G4-129S6B6F1 cell line by PCR using primer 5′-RAForward (5′-CTA GCG GAT CCC GGG TGG GGG ACA GAG TGC AAA CTA GGT AGA TAC-3′)(SEQ ID NO: 9) and primer 3′-RA Reverse (5′-ATT TGG AGC TCG TCG ACC TCA GTG TGT CTC AGG CTT G-3′)(SEQ ID NO: 10). The resulting fragment was purified as described above, sequenced and digested with SacI and BamHI restriction endonucleases and ligated into a pBS-Bluescript SacI/BamHI vector.

The long arm (LA) (DNA sequence from ENSMUSG00000047259) was amplified from genomic DNA extracted from an ES G4-129S6B6F1 cell line by PCR using primer LA#3 Forward (5′-GGG TAC CGT CGA CAA GCG AGG GAA CAG GGT CTC CAT AGA GAC-3′)(SEQ ID NO: 11) and primer 3′-LA Reverse (5′-GGA GTG GAT CCT TCC TGC AGC AGC TGG ATT TGA GTC CTC C-3′)(SEQ ID NO: 12) and the resulting fragment was purified as described above, sequenced and digest with KpnI and BamHI restriction endonucleases and ligated into a pBS-Bluescript-RA KpnI/BamHI vector.

In order to flank the Neomycin selection cassette by FRT sites, PCR was performed from a pHR56 Neo plasmid vector (described in Metzger D. et al, Proc. Natl. Acad. Sci. USA Vol. 92, pp. 6991-6995, July 1995) using 5′-NeoFRT Forward primer (5′-ATA TCA AGC TTG AAG TTC CTA TAC TTT CTA GAG AAT AGG AAC TTC TAC CGG GTA GGG GAG GCG CTT TTC CCA AGG-3′)(SEQ ID NO: 13) and 3′-NeoFRT Reverse primer (5′-AGC TGC CCG GGA AGT TCC TAT TCT CTA GAA AGT ATA GGA ACT TCA GCT TCT GAT GGA ATT AGA ACT TGG CAA AAC-3′)(SEQ ID NO: 14). The resulting fragment was purified as described above, sequenced and digested with HindIII and SmaI restriction endonucleases, and ligated into a pBK-CMV HindIII/SmaI vector.

In order to flank the Venus coding sequence by LoxP sites, PCR was performed from a pcDNA3.1-Venus Zeo (+) vector (kindly provided by Dr. Miyawaki) using 5′-VenLOX Forward primer (5′-TCT TTG GAT CCG CGG ATA ACT TCG TAT AGC ATA CAT TAT ACG AAG TTA TCC ATG GTG AGC AAG GGC GAG GAG CTG TTC ACC G-3′)(SEQ ID NO: 15) and 3′-VenLOX Reverse primer (5′-TCA AAA AGC TTA TAA CTT CGT ATA ATG TAT GCT ATA CGAAGT TAT CTA CTT GTA CAG CTC GTC CAT GCC GAG AGT G-3′)(SEQ ID NO: 16). The resulting fragment was purified as described above, sequenced and digested with BamHI and HindIII restriction endonucleases and ligated into a pBK-CMV-Neo BamHI/HindIII vector.

The fragment 3HA-hMC4R(R165W) or myc-hMC4R(WT) BamHI/SacII was then ligated to the plasmid pBK-CMV-Neo-Venus BamHI/SacII. The plasmid pBK-CMV-3HA-hMC4R(R165W)-Venus-Neo or myc-hMC4R(WT)-Venus-Neo was digested with BamHI and SmaI restriction endonucleases. The fragment 3HA-hMC4R(R165W)-Venus-Neo or myc-hMC4R(WT)-Venus-Neo BamHI/SmaI was ligated to the plasmid pBS-Bluescript-LA-RA, which was cleaved beforehand with BamHI and SmaI restriction endonucleases to obtain the targeting vector.

The plasmid was used to transform E. coli and amplified. Plasmid purification was done using a QIAGEN maxiprep kit (QIAGEN Mississauga, ON, Canada). The plasmid was cleaved by Sal I to linearize the targeting vector before electroporation in ES G4-129S6B6F1 cell lines.

All PCR products were sequenced to confirm the presence of desired modifications and absence of unwanted mutations.

Generation of hMC4R Knock-in Mice

The targeting construct consisting of 8.6 kb was electroporated into ES G4-129S6B6F1 cell lines. Targeted clones were identified by Southern blot analysis using ApaI digestion of ES cell genomic DNA and a labeled PCR-amplified DNA fragment derived from a flanking region 3′ of the targeting construct as a hybridization probe (probe C). Cells selected for homologous recombination at the mMC4R locus were then electroporated with a plasmid coding for the Flip recombinase to excise the neomycin cassette prior to transfer of ES positive cells to blastocysts. New ES clones were screened by Southern blot hybridization analysis of SacI-digested-genomic DNA with the flanking probe C. ES clones with the predicted pattern were injected into C57BL6 blastocysts and germline-transmitting chimeric animals were obtained and then mated with C57BL6 mice. The resulting heterozygous offspring were crossed to generate non-transgenic littermates, heterozygous, and homozygous hMC4R Knock-in mice. All mice were thus on a mixed C57Bl6/J and 129Sv background. Offspring were genotyped using the same strategy as for selecting ES neo-excised clones by Southern blot analysis.

### Southern Blot Hybridization

Genomic DNA from an ES G4-129S6B6F1 cell line or tail biopsies was prepared using a tissue DNA extraction kit (eZNA, D3396-02, OMEGA bio-tek, Norcross, Ga., USA). 20 ug of genomic DNA was digested overnight with the indicated restriction endonucleases, and electrophoresed through a 0.8% agarose gel. The digested DNA was subsequently transferred to an Amersham Hybond N⁺ nylon membrane (GE Healthcare: # RPN203B) by a capillary transfer method and hybridized with a ³²P-radiolabeled probe of 500 bp. The flanking Probe C at the mMC4R locus was amplified from genomic DNA extracted from an ES G4-129S6B6F1 cell line by PCR (annealing temperature 60° C., 30 cycles) using primer C forward: 5′-GGG CAT CCA TGT GCA AAT CCG TAT CAA AGT-3′ (SEQ ID NO: 17) and primer C reverse: 5′-GGG CCC AAG CAC AGA CCC ATG TAT AAT TC-3′ (SEQ ID NO: 18). The resulting fragment was purified as described above.

The probe was labeled with ³²P-dCTP using the DECAprime II Random Primed DNA Labeling Kit (Ambion, Inc., Austin, Tex., USA) according to the manufacturer's instructions. Hybridization was performed in Ultrahyb Hybridization buffer (Ambion, Inc., Austin, Tex., USA) and 10⁶ cpm/ml of denaturated probe overnight at 42° C. The membrane was washed by successive washes in 2×SSC/0.1% SDS for 20 minutes at 50° C., 2×SSC/0.1% SDS for 20 minutes at 55° C. and 0.2×SSC/0.1% SDS for 20 minutes at 60° C., and exposed to X-ray film for 48 hrs at −80° C.

### Production of Knock-in Mice and Animal Care

Using standard ES cell procedures, chimeric animals were obtained and mated with C57BL6 mice to generate mice heterozygous for either the 3HA-hMC4R(R165W)-Venus or myc-hMC4R(WT)-Venus allele on a mixed C57Bl6/J and 129S6B6F1 background.

Animals were housed under specific-pathogen-free conditions and were handled in accordance with procedures and protocols approved by Université de Montreal institutional animal care committees. Mice were housed in groups of two to five mice at 22° C.-24° C. using a 12 hr light/12 hr dark cycle (6:00 am-6:00 pm) with chow food (Teklad global 18% protein diet 2028, 3.1 kcal/g metabolizable energy, 18% kcal from fat, Harlan Teklad, Madison, Wis.) and water provided ad libitum.

Mice maintained on a mixed C57Bl6/J and 129S6B6F1 genetic background were used for initial phenotypic characterization, histology analysis, and growth studies.

### Immunohistochemistry

Brains from transgenic animals were collected and snap-frozen in isopentane and stored at −80° C. until further processing. Sections were cut at 10 μm using a cryostat. Serial 10-μm thick frozen brain sections were then processed in the automatic Discovery XT Ventana Med System for indirect peroxidase labeling using as primary antibody anti-GFP (Ab290 from abcam at 1:1000) against the C-terminal yellow fluorescent protein fused to the transgene receptor. Tissue sections were then stained using a conventional hematoxylin and eosin protocol.

### Phenotyping

At three weeks of age, mice were weaned and group-housed with littermates of the same sex. At 4 weeks of age, weight gain was measured regularly on a weekly basis until 16 weeks of age. Basal food intake on regular chow diet (Teklad Global 18% Protein Rodent Diet from Harlan laboratories) was measured in individualized mice of 18-22 weeks of age. Mice were individually housed at least four days before any measurements were taken. A sufficient amount of food for the week was then weighed and provided to the mice ad libitum. Each day, morning and afternoon at the same time, the remaining food was measured for 5 consecutive days. The daily average of food intake during dark cycle and light cycle was calculated for each genotype.

Necropsies were performed at 18-22 weeks of age for measuring fat mass content. Abdominal fat and subcutaneous fat were dissected from each mouse and weighed. Length was measured on anaesthetized mice (with Isoflurane 2%) by manual extension of the mouse to its full length and measurement of the nose-to-anus distance in centimeters.

### DCPMP Treatment

Animals were individually housed for 4 days prior to starting experiments. Knock-in mice and littermate controls had basal feeding monitored during dark and light cycles for 4 days, and then for 3 days following an intraperitoneal (i.p.) saline injection to demonstrate that the observed effects were not due to differential stress responses. All animals were weighed prior to compound injection, and doses were normalized to individual animal body weight. Mice were intraperitoneally injected one dose daily with vehicle (1% Tween 80) or DCPMP at 30 mg per kg one hour before lights off (at 5:00 PM), for 9 days. Weight was monitored every 3 days during treatment and after stopping treatment. Food intake was measured during the recovery time for 7 days. Data are the average daily food intake or the mean of total body weight loss [total body weight before treatment—total body weight after 9 days treatment].

The contents of all documents and references cited herein are hereby incorporated by reference in their entirety, to the same extent as if each individual document or reference was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A transgenic non-human animal, cell, or tissue comprising in its genome a transgene encoding a mutated human melanocortin type-4 receptor (hMC4R) protein, wherein the mutated hMC4R protein promotes obesity.

2. (canceled)

3. (canceled)

4. The transgenic non-human animal, cell, or tissue of claim 1, wherein the mutated hMC4R protein comprises an arginine at position 165 of the hMC4R protein in place of a tryptophan (R165W mutation).

5. The transgenic non-human animal, cell, or tissue of claim 1, wherein the animal, cell, or tissue is heterozygous or homozygous for the mutated hMC4R protein.

6. The transgenic non-human animal, cell, or tissue of claim 1, wherein the endogenous animal, cell, or tissue MC4R gene is functionally disrupted or deleted and replaced by the transgene encoding the mutated hMC4R protein.

7. The transgenic non-human animal, cell, or tissue of claim 1, wherein the transgene further comprises a detectable marker or tag selected from a fluorescent protein, a human influenza hemagglutinin (HA) tag, and a myc tag, and/or a site-specific recombinase system.

8.-13. (canceled)

14. The transgenic non-human animal, cell, or tissue of claim 7, wherein the transgene comprises a yellow fluorescent protein encoded by a Venus gene sequence, and the Venus gene sequence is flanked by LoxP sites, allowing removal of the yellow fluorescent protein in the transgenic non-human animal, cell, or tissue.

15. The transgenic non-human animal, cell, or tissue of claim 14, wherein the transgene further comprises a neomycin cassette flanked by FRT sites.

16. The transgenic non-human animal, cell, or tissue of claim 1, wherein the transgene is inserted into the animal, cell, or tissue genome via homologous recombination.

17.-19. (canceled)

20. The transgenic non-human animal, cell, or tissue of claim 1, wherein the transgenic non-human animal, cell, or tissue has symptoms of MC4R-induced obesity selected from obesity, hyperphagia, increased fat mass, increased linear growth, and/or obesity associated metabolic disorders, relative to a nontransgenic non-human animal, cell, or tissue.

21-39. (canceled)

40. A transgene encoding a mutated human melanocortin type-4 receptor (hMC4R) protein, wherein the mutated hMC4R protein promotes obesity.

41. The transgene of claim 40, wherein the mutated hMC4R protein is non-functional, improperly folded compared to wild-type hMC4R protein, and/or retained intracellularly.

42. (canceled)

43. The transgene of claim 40, wherein the mutated hMC4R protein comprises an arginine at position 165 of the hMC4R protein in place of a tryptophan (R165W mutation).

44. The transgene of claim 40, wherein the transgene further comprises a detectable marker or tag selected from a fluorescent protein, a human influenza hemagglutinin (HA) tag, and a myc tag, and/or a site-specific recombinase system.

45-50. (canceled)

51. The transgene of claim 44, wherein the transgene comprises a yellow fluorescent protein encoded by a Venus gene sequence, and the Venus gene sequence is flanked by LoxP sites.

52. The transgene of claim 51, further comprising a neomycin cassette flanked by FRT sites and/or sequences for insertion into a host chromosome at the MC4R locus via homologous recombination.

53. (canceled)

54. (canceled)

55. A method of screening for an agent for treating obesity or for treating MC4R deficiency, comprising:
providing a transgenic non-human animal comprising the transgene of claim 40, wherein the transgene is expressed to produce the mutated human MC4R protein;
administering the agent to the transgenic non-human animal; and
determining level of obesity in the transgenic non-human animal;
wherein a reduced level of obesity or obesity-associated metabolic disorders in the transgenic non-human animal compared to the level of obesity or obesity-associated metabolic disorders in a control transgenic non-human animal which is not administered the agent indicates the agent is for use for treating obesity or MC4R deficiency.

56. (canceled)

57. The method of claim 55, further comprising determining cell surface expression and/or signaling activity of the mutated hMC4R protein, wherein an increase in cell surface expression and/or signaling activity of the mutated hMC4R protein after treatment with the agent, compared to the control transgenic non-human animal, indicates that the agent is for use for treating obesity or MC4R deficiency.

58.-104. (canceled)

105. The transgenic non-human animal, cell, or tissue of claim 1, wherein the mutated hMC4R protein encoded by the transgene has the amino acid sequence set forth in SEQ ID NO: 25 or SEQ ID NO: 27, or a sequence substantially identical thereto, or a variant thereof.

106. The transgenic non-human animal, cell, or tissue of claim 1, wherein the transgene encoding the mutated hMC4R protein comprises the sequence set forth in SEQ ID NOs: 19, 21, 23, or 29, or a sequence substantially identical thereto, or a variant thereof.

107.-109. (canceled)

110. The transgene of claim 40 or the method of claim 55, wherein the transgene comprises the sequence set forth in SEQ ID NOs: 19, 21, 23, or 29, or a sequence substantially identical thereto, or a variant thereof; or the mutated hMC4R protein encoded by the transgene has the amino acid sequence set forth in SEQ ID NO: 25 or SEQ ID NO: 27, or a sequence substantially identical thereto, or a variant thereof.

111.-121. (canceled)


SEQUENCE LISTING (30 sequences)

SEQ ID NO: 1 - DNA, 22 bp, Artificial Sequence
  primer
attaatacgactcactataggg

SEQ ID NO: 2 - DNA, 24 bp, Artificial Sequence
  primer
agaacgtggactccaacgtcaaag

SEQ ID NO: 3 - DNA, 25 bp, Artificial Sequence
  primer
gacagttaagtgggttgggatcatc

SEQ ID NO: 4 - DNA, 84 bp, Artificial Sequence
  primer
tcggatccccgagatctcaccatggcatcaatgcagaagctgatctcagaggaggacctgaattcggtgaactccacccaccgt

SEQ ID NO: 5 - DNA, 32 bp, Artificial Sequence
  primer
taagcttggatccatgtacccatacgatgttc

SEQ ID NO: 6 - DNA, 33 bp, Artificial Sequence
  primer
ccatgggatccatgcagaagctgatctcagagg

SEQ ID NO: 7 - DNA, 30 bp, Artificial Sequence
  primer
gttgtctgctgggcaccattcttcctccac

SEQ ID NO: 8 - DNA, 42 bp, Artificial Sequence
  primer
cctccccgcggatacctgctagacaagtcacaaaggcctccc

SEQ ID NO: 9 - DNA, 45 bp, Artificial Sequence
  primer
ctagcggatcccgggtgggggacagagtgcaaactaggtagatac

SEQ ID NO: 10 - DNA, 37 bp, Artificial Sequence
  primer
atttggagctcgtcgacctcagtgtgtctcaggcttg

SEQ ID NO: 11 - DNA, 42 bp, Artificial Sequence
  primer
gggtaccgtcgacaagcgagggaacagggtctccatagagac

SEQ ID NO: 12 - DNA, 40 bp, Artificial Sequence
  primer
ggagtggatccttcctgcagcagctggatttgagtcctcc

SEQ ID NO: 13 - DNA, 75 bp, Artificial Sequence
  primer
atatcaagcttgaagttcctatactttctagagaataggaacttctaccgggtaggggaggcgcttttcccaagg

SEQ ID NO: 14 - DNA, 75 bp, Artificial Sequence
  primer
agctgcccgggaagttcctattctctagaaagtataggaacttcagcttctgatggaattagaacttggcaaaac

SEQ ID NO: 15 - DNA, 82 bp, Artificial Sequence
  primer
tctttggatccgcggataacttcgtatagcatacattatacgaagttatccatggtgagcaagggcgaggagctgttcaccg

SEQ ID NO: 16 - DNA, 76 bp, Artificial Sequence
  primer
tcaaaaagcttataacttcgtataatgtatgctatacgaagttatctacttgtacagctcgtccatgccgagagtg

SEQ ID NO: 17 - DNA, 30 bp, Artificial Sequence
  primer
gggcatccatgtgcaaatccgtatcaaagt

SEQ ID NO: 18 - DNA, 29 bp, Artificial Sequence
  primer
gggcccaagcacagacccatgtataattc

SEQ ID NO: 19 - DNA, 8655 bp, Artificial Sequence
  Full transgene sequence (R165W)
cgtcgacaagtttgttatgggggaaggttgatagagattaatctaatttaattattttaatataaaaggtaaggatgagaaatagtcttagtaaaatttctaaagtaagggctagtgaaaagtctctcacccagtaagtgcacaccactagcaacatttgttagcattatactgtacacaacatgaaacaaactaactaatgtaccaagcaatttaggaagaagccagtaaatcactaaattacagaacgtttaaaaagtaaactttgtggtatttaaccagcttggtttatatccttgaaaatcaactgggagaaagatactaaaaagcatctaccaggaagtatagaggcaacctgacttgaaagtccttagaaacctcttatcacctttacggagaagcaactcagcttacctgaattgaaccccccaaaccagacttctaagtctttcttctgacccagaatgtgctctctaagaagagacttgacccccattggctctagaagcaactgttaatctcatggttacctgacattttgggcacaacttagagaaaagaatttaaaaatttaaaggcaagcgagggaacagggtctccatagagacagatcgaatgagctccgagaatataaaaagtcgtgctgtttgtagagctatgcataaggtcatgaaagagccaagagtgccctaagtcctcacctttggctgatttctcctctgtgcaaacagagagtgaggggaggggaggctgccaagtgagagtggggtgggggtggggtgacacaatgtaggcttgcttcaacacacaaaactaggagacttcctgtttgtagctgccttcgtccatcactctctgaccagtaagatcacaaatcagatggtttattgggcacatgagacagaccagtacaaacttttctcgtaagtcactgaaataaacaacaatgataaccaaccacggtaacaaaaaaaaaaaaaaatgtaagcagtaaatatggtttccagaatagctgcaacatacttaatatgtccagtttggaacaaaaataaaaagaagtaggaaaggaaataagaaagtatgaccatacacagaagaaaaaaaaaactgacctttaaaagttattcttggggaaagccactgtgtgtgaaattctttaagccagttattatgagcacctcatatatatcacacctataggagtaacattccatcacaggcaattttaataaagcaatataagccttcatgatgctgggttaggaaagactttctttgctatgaaaaccagatgcactctatgcccaagggcaagagttactcagaaagattattcagttaaccacgtgatacttaaacaactccacggagttcttctctatcacttatttactggttagataattcgccattggagaaagtgtgaaaaggaacatgaatatgtaaatcatccttcttaagggctttagtgaagtgtctggagggaacaactgccagctgctgtaacagctgagccacttgggagtttacaatagtaaaagctgccccttgttcctagcgcatttgcggtcgttggtcagctcaagatggaatgagtctagagggggagctctattctgcacagttacttagtagggggcttctacaccacagagcatctagaaactctgcagtcattttccaaacaggagaaaaagtataatcaagagaaaggtgacgcaatgagtaccaactcctgtactattagtcagaattcagtagtaggtcctactcagttcctgagttgcatggcaacactaacttagcacagtgttctgaagaaacggttagcattgtctctaacacacacacacacacacacacacacacacacacacacacacacacacacggggtgcgggggggaatgggggcgggggcggggctcaaagctaagtggccatctcttcaaagcaacacacatgaatgtgcactcactggagctagcctgagtaagtacggagccaaagtgtttacagtttcaggttgtgatgcttccaactatggaagaagaaaaataaacaagttaaaaaacaaaaaccagaacactatctttaaaaaaaaaaacaaacaacaaaacatcaacaaaatcctaccaacctaaccaacaataacaacaacagaccatgcagaatattatcacattgttgatttgtctaagttcacagacaatacgctttcacaaatgccacttgaactttttaaaatacagtgaatgaccacttttccagccgttagagcagaaactgcaaatggagaaacagctaccagcacgcaagaagtgaacctggtcttaatctctggctttgctgtaattctccacagcatactcatctatttaattaaattactgcctgcatttctagtctttcaaactgagccttccgtcattcaggggggcatcctgggcagaggggcagcgcctgcttcgggagccaggacagctgcttttcatttcttttttttatccacaatcacgcatgagttcagtctcaaggaggaaagagcctggctgattcccgaggatttcaggagtatctcagaatgtgctcgagcaatcaagtcatttctcttaaaacttggaaaggaaaatcccgtgataccttcccaccccagcagccctagccactgagccggttgctgcgctgtaatctatctgtgcaagatcgatgtctcagaaccactgaatacggattggtcagaaggaagcagaggaggagccattcagaacaccccccaccccccggcccccctccgtctaaccataagaaatcagcagcccgcactaggctgctctggctcacaaagatgctcaggaagctgaacttctgagaggctgcggtgtgagtgtgggcgcgcagatgcagacgcggctcccagcagtacagcgagtctcagggaaaaggactctgaaaagaccccgagtgaatactaaagtgaaagccgcactgagagagagagaaaaaaaagcaaacagcagactggtcaaccgagaatgagcattcagaagcaccagcttctaaagagacgatgatctgagccgaacccagaagagaccaacaactcctttgcgagttccgctgcttctgaccctgctcctagcaggcgccaagcgcagcctcccaacttctacaggcatacagactgggagagaatcactcggagcttccctgacccaggaggttggatcagttcaaggaggactcaaatccagctgctgcaggaaggatccatgtacccatacgatgttccagattacgcttacccatacgatgttccagattacgcttacccatacgatgttccagattacgctgatgtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagtgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggcacattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcaggtatccgcggataacttcgtatagcatacattatacgaagttatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtagataacttcgtatagcatacattatacgaagttataagcttgaagttcctatactttctagagaataggaacttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgaagttcctatactttctagagaataggaacttcccgggtgggggacagagtgcaaactaggtagatacctgcagactttgtcactctggcccgatctgagcagtgtacttcccaacagctgcctcttctgtgtaatgctttggttgaaaatatctactgtataaatgtaagtttgtgacttttgacatggaaaaaaaagtctcaacgtgttatgtttattgacacgctattttttttgtttgtaaaatgcttatttatgttctatatagtgtgggcgttatgaattgacatgaaagaaaaacagacacccttattaaaactttgacagtgtttctttcctgttatttatcaaggttccacacttgttctttctgtagtggccgaaatcagaaccttattaaacgtgttctcagctgttctcatgtattagccccacagtactgcagaggcactgaccccactgtttatggggaaatatttaaacactacatgcttgatcattaaaatgagtcagctctcttagtgaaatttcgagcaatcgaataaaagcttgcctattatccttgctgtccaaatacactgatgcttctttttaagtaaaggaaagagaaagggggaagaagcagctactgaggagaaagtgagatttctgtcacatgcatttctccaagaaggaatggttcattgcccgagactcagagttcacacaggcaagtcagctgtggtaggggaaatgcccacttaatagattaaagatattataatagataataatagataaaatagattaatagataaaatagataccaatcttaatagattaaagtgtcctgttaaatataaactgtccacaccatgctgaaatttcctatgccaaatgataccccaccataacagaatgatttctttctggcttcttaccagggatctggttcctacagaaaggtctagaacagctccctctgcacttagaggtccagcgttcatttcatcttagagttaatagtgagttgtgctatctttcatgtggcgggggacttgttgttcactttctgattactttttgagctggaatataagtgctgaagatcaagtgatttaattcccaagccaaatccacatcacaaaacattttgggacagggtttgtaaatatctaaagtgtggagccctgtggtgcttgcacataacgagatggaaagagaacacaaatggggtcctggaaggtacagtaaaacaccctgctgttcttagtcatgtcttgggatggggaatgcttgttttctccaaactaataccaaaggtgtggccactgagcaaccaaatctatgctttctagtctgtgtatactttgaaataaaagggataaaaactacattaggcctatgactagaggccaactttcctgtgattcaattgatagtgtctcccaatgcgcataggcagagacaatgcatagcagtgcatctgaattaaaacctgaatgttccacctattaattacaagcctgagacacactgagg

SEQ ID NO: 20 - DNA, 8605 bp, Artificial Sequence
  Full transgene sequence (wild type)
cgtcgacaagtttgttatgggggaaggttgatagagattaatctaatttaattattttaatataaaaggtaaggatgagaaatagtcttagtaaaatttctaaagtaagggctagtgaaaagtctctcacccagtaagtgcacaccactagcaacatttgttagcattatactgtacacaacatgaaacaaactaactaatgtaccaagcaatttaggaagaagccagtaaatcactaaattacagaacgtttaaaaagtaaactttgtggtatttaaccagcttggtttatatccttgaaaatcaactgggagaaagatactaaaaagcatctaccaggaagtatagaggcaacctgacttgaaagtccttagaaacctcttatcacctttacggagaagcaactcagcttacctgaattgaaccccccaaaccagacttctaagtctttcttctgacccagaatgtgctctctaagaagagacttgacccccattggctctagaagcaactgttaatctcatggttacctgacattttgggcacaacttagagaaaagaatttaaaaatttaaaggcaagcgagggaacagggtctccatagagacagatcgaatgagctccgagaatataaaaagtcgtgctgtttgtagagctatgcataaggtcatgaaagagccaagagtgccctaagtcctcacctttggctgatttctcctctgtgcaaacagagagtgaggggaggggaggctgccaagtgagagtggggtgggggtggggtgacacaatgtaggcttgcttcaacacacaaaactaggagacttcctgtttgtagctgccttcgtccatcactctctgaccagtaagatcacaaatcagatggtttattgggcacatgagacagaccagtacaaacttttctcgtaagtcactgaaataaacaacaatgataaccaaccacggtaacaaaaaaaaaaaaaaatgtaagcagtaaatatggtttccagaatagctgcaacatacttaatatgtccagtttggaacaaaaataaaaagaagtaggaaaggaaataagaaagtatgaccatacacagaagaaaaaaaaaactgacctttaaaagttattcttggggaaagccactgtgtgtgaaattctttaagccagttattatgagcacctcatatatatcacacctataggagtaacattccatcacaggcaattttaataaagcaatataagccttcatgatgctgggttaggaaagactttctttgctatgaaaaccagatgcactctatgcccaagggcaagagttactcagaaagattattcagttaaccacgtgatacttaaacaactccacggagttcttctctatcacttatttactggttagataattcgccattggagaaagtgtgaaaaggaacatgaatatgtaaatcatccttcttaagggctttagtgaagtgtctggagggaacaactgccagctgctgtaacagctgagccacttgggagtttacaatagtaaaagctgccccttgttcctagcgcatttgcggtcgttggtcagctcaagatggaatgagtctagagggggagctctattctgcacagttacttagtagggggcttctacaccacagagcatctagaaactctgcagtcattttccaaacaggagaaaaagtataatcaagagaaaggtgacgcaatgagtaccaactcctgtactattagtcagaattcagtagtaggtcctactcagttcctgagttgcatggcaacactaacttagcacagtgttctgaagaaacggttagcattgtctctaacacacacacacacacacacacacacacacacacacacacacacacacacggggtgcgggggggaatgggggcgggggcggggctcaaagctaagtggccatctcttcaaagcaacacacatgaatgtgcactcactggagctagcctgagtaagtacggagccaaagtgtttacagtttcaggttgtgatgcttccaactatggaagaagaaaaataaacaagttaaaaaacaaaaaccagaacactatctttaaaaaaaaaaacaaacaacaaaacatcaacaaaatcctaccaacctaaccaacaataacaacaacagaccatgcagaatattatcacattgttgatttgtctaagttcacagacaatacgctttcacaaatgccacttgaactttttaaaatacagtgaatgaccacttttccagccgttagagcagaaactgcaaatggagaaacagctaccagcacgcaagaagtgaacctggtcttaatctctggctttgctgtaattctccacagcatactcatctatttaattaaattactgcctgcatttctagtctttcaaactgagccttccgtcattcaggggggcatcctgggcagaggggcagcgcctgcttcgggagccaggacagctgcttttcatttcttttttttatccacaatcacgcatgagttcagtctcaaggaggaaagagcctggctgattcccgaggatttcaggagtatctcagaatgtgctcgagcaatcaagtcatttctcttaaaacttggaaaggaaaatcccgtgataccttcccaccccagcagccctagccactgagccggttgctgcgctgtaatctatctgtgcaagatcgatgtctcagaaccactgaatacggattggtcagaaggaagcagaggaggagccattcagaacaccccccaccccccggcccccctccgtctaaccataagaaatcagcagcccgcactaggctgctctggctcacaaagatgctcaggaagctgaacttctgagaggctgcggtgtgagtgtgggcgcgcagatgcagacgcggctcccagcagtacagcgagtctcagggaaaaggactctgaaaagaccccgagtgaatactaaagtgaaagccgcactgagagagagagaaaaaaaagcaaacagcagactggtcaaccgagaatgagcattcagaagcaccagcttctaaagagacgatgatctgagccgaacccagaagagaccaacaactcctttgcgagttccgctgcttctgaccctgctcctagcaggcgccaagcgcagcctcccaacttctacaggcatacagactgggagagaatcactcggagcttccctgacccaggaggttggatcagttcaaggaggactcaaatccagctgctgcaggaaggatccatgcagaagctgatctcagaggaggacctgaattcggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggcaccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcaggtatccgcggataacttcgtatagcatacattatacgaagttatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtagataacttcgtatagcatacattatacgaagttataagcttgaagttcctatactttctagagaataggaacttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgaagttcctatactttctagagaataggaacttcccgggtgggggacagagtgcaaactaggtagatacctgcagactttgtcactctggcccgatctgagcagtgtacttcccaacagctgcctcttctgtgtaatgctttggttgaaaatatctactgtataaatgtaagtttgtgacttttgacatggaaaaaaaagtctcaacgtgttatgtttattgacacgctattttttttgtttgtaaaatgcttatttatgttctatatagtgtgggcgttatgaattgacatgaaagaaaaacagacacccttattaaaactttgacagtgtttctttcctgttatttatcaaggttccacacttgttctttctgtagtggccgaaatcagaaccttattaaacgtgttctcagctgttctcatgtattagccccacagtactgcagaggcactgaccccactgtttatggggaaatatttaaacactacatgcttgatcattaaaatgagtcagctctcttagtgaaatttcgagcaatcgaataaaagcttgcctattatccttgctgtccaaatacactgatgcttctttttaagtaaaggaaagagaaagggggaagaagcagctactgaggagaaagtgagatttctgtcacatgcatttctccaagaaggaatggttcattgcccgagactcagagttcacacaggcaagtcagctgtggtaggggaaatgcccacttaatagattaaagatattataatagataataatagataaaatagattaatagataaaatagataccaatcttaatagattaaagtgtcctgttaaatataaactgtccacaccatgctgaaatttcctatgccaaatgataccccaccataacagaatgatttctttctggcttcttaccagggatctggttcctacagaaaggtctagaacagctccctctgcacttagaggtccagcgttcatttcatcttagagttaatagtgagttgtgctatctttcatgtggcgggggacttgttgttcactttctgattactttttgagctggaatataagtgctgaagatcaagtgatttaattcccaagccaaatccacatcacaaaacattttgggacagggtttgtaaatatctaaagtgtggagccctgtggtgcttgcacataacgagatggaaagagaacacaaatggggtcctggaaggtacagtaaaacaccctgctgttcttagtcatgtcttgggatggggaatgcttgttttctccaaactaataccaaaggtgtggccactgagcaaccaaatctatgctttctagtctgtgtatactttgaaataaaagggataaaaactacattaggcctatgactagaggccaactttcctgtgattcaattgatagtgtctcccaatgcgcataggcagagacaatgcatagcagtgcatctgaattaaaacctgaatgttccacctattaattacaagcctgagacacactgagg

SEQ ID NO: 21 - DNA, 999 bp, Artificial Sequence
  Sequence encoding R165W MC4R protein
atggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagtgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcagatattaa

SEQ ID NO: 22 - DNA, 999 bp, Artificial Sequence
  Sequence encoding wild type MC4R protein
atggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcagatattaa

SEQ ID NO: 23 - DNA, 6676 bp, Artificial Sequence
  transgene with neo excised (R165W)
cgtcgacaagtttgttatgggggaaggttgatagagattaatctaatttaattattttaatataaaaggtaaggatgagaaatagtcttagtaaaatttctaaagtaagggctagtgaaaagtctctcacccagtaagtgcacaccactagcaacatttgttagcattatactgtacacaacatgaaacaaactaactaatgtaccaagcaatttaggaagaagccagtaaatcactaaattacagaacgtttaaaaagtaaactttgtggtatttaaccagcttggtttatatccttgaaaatcaactgggagaaagatactaaaaagcatctaccaggaagtatagaggcaacctgacttgaaagtccttagaaacctcttatcacctttacggagaagcaactcagcttacctgaattgaaccccccaaaccagacttctaagtctttcttctgacccagaatgtgctctctaagaagagacttgacccccattggctctagaagcaactgttaatctcatggttacctgacattttgggcacaacttagagaaaagaatttaaaaatttaaaggcaagcgagggaacagggtctccatagagacagatcgaatgagctccgagaatataaaaagtcgtgctgtttgtagagctatgcataaggtcatgaaagagccaagagtgccctaagtcctcacctttggctgatttctcctctgtgcaaacagagagtgaggggaggggaggctgccaagtgagagtggggtgggggtggggtgacacaatgtaggcttgcttcaacacacaaaactaggagacttcctgtttgtagctgccttcgtccatcactctctgaccagtaagatcacaaatcagatggtttattgggcacatgagacagaccagtacaaacttttctcgtaagtcactgaaataaacaacaatgataaccaaccacggtaacaaaaaaaaaaaaaaatgtaagcagtaaatatggtttccagaatagctgcaacatacttaatatgtccagtttggaacaaaaataaaaagaagtaggaaaggaaataagaaagtatgaccatacacagaagaaaaaaaaaactgacctttaaaagttattcttggggaaagccactgtgtgtgaaattctttaagccagttattatgagcacctcatatatatcacacctataggagtaacattccatcacaggcaattttaataaagcaatataagccttcatgatgctgggttaggaaagactttctttgctatgaaaaccagatgcactctatgcccaagggcaagagttactcagaaagattattcagttaaccacgtgatacttaaacaactccacggagttcttctctatcacttatttactggttagataattcgccattggagaaagtgtgaaaaggaacatgaatatgtaaatcatccttcttaagggctttagtgaagtgtctggagggaacaactgccagctgctgtaacagctgagccacttgggagtttacaatagtaaaagctgccccttgttcctagcgcatttgcggtcgttggtcagctcaagatggaatgagtctagagggggagctctattctgcacagttacttagtagggggcttctacaccacagagcatctagaaactctgcagtcattttccaaacaggagaaaaagtataatcaagagaaaggtgacgcaatgagtaccaactcctgtactattagtcagaattcagtagtaggtcctactcagttcctgagttgcatggcaacactaacttagcacagtgttctgaagaaacggttagcattgtctctaacacacacacacacacacacacacacacacacacacacacacacacacacggggtgcgggggggaatgggggcgggggcggggctcaaagctaagtggccatctcttcaaagcaacacacatgaatgtgcactcactggagctagcctgagtaagtacggagccaaagtgtttacagtttcaggttgtgatgcttccaactatggaagaagaaaaataaacaagttaaaaaacaaaaaccagaacactatctttaaaaaaaaaaacaaacaacaaaacatcaacaaaatcctaccaacctaaccaacaataacaacaacagaccatgcagaatattatcacattgttgatttgtctaagttcacagacaatacgctttcacaaatgccacttgaactttttaaaatacagtgaatgaccacttttccagccgttagagcagaaactgcaaatggagaaacagctaccagcacgcaagaagtgaacctggtcttaatctctggctttgctgtaattctccacagcatactcatctatttaattaaattactgcctgcatttctagtctttcaaactgagccttccgtcattcaggggggcatcctgggcagaggggcagcgcctgcttcgggagccaggacagctgcttttcatttcttttttttatccacaatcacgcatgagttcagtctcaaggaggaaagagcctggctgattcccgaggatttcaggagtatctcagaatgtgctcgagcaatcaagtcatttctcttaaaacttggaaaggaaaatcccgtgataccttcccaccccagcagccctagccactgagccggttgctgcgctgtaatctatctgtgcaagatcgatgtctcagaaccactgaatacggattggtcagaaggaagcagaggaggagccattcagaacaccccccaccccccggcccccctccgtctaaccataagaaatcagcagcccgcactaggctgctctggctcacaaagatgctcaggaagctgaacttctgagaggctgcggtgtgagtgtgggcgcgcagatgcagacgcggctcccagcagtacagcgagtctcagggaaaaggactctgaaaagaccccgagtgaatactaaagtgaaagccgcactgagagagagagaaaaaaaagcaaacagcagactggtcaaccgagaatgagcattcagaagcaccagcttctaaagagacgatgatctgagccgaacccagaagagaccaacaactcctttgcgagttccgctgcttctgaccctgctcctagcaggcgccaagcgcagcctcccaacttctacaggcatacagactgggagagaatcactcggagcttccctgacccaggaggttggatcagttcaaggaggactcaaatccagctgctgcaggaaggatccatgtacccatacgatgttccagattacgcttacccatacgatgttccagattacgcttacccatacgatgttccagattacgctgatgtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagtgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggcaccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcaggtatccgcggataacttcgtatagcatacattatacgaagttatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtagataacttcgtatagcatacattatacgaagttataagcttgaagttcctatactttctagagaataggaacttcccgggtgggggacagagtgcaaactaggtagatacctgcagactttgtcactctggcccgatctgagcagtgtacttcccaacagctgcctcttctgtgtaatgctttggttgaaaatatctactgtataaatgtaagtttgtgacttttgacatggaaaaaaaagtctcaacgtgttatgtttattgacacgctattttttttgtttgtaaaatgcttatttatgttctatatagtgtgggcgttatgaattgacatgaaagaaaaacagacacccttattaaaactttgacagtgtttctttcctgttatttatcaaggttccacacttgttctttctgtagtggccgaaatcagaaccttattaaacgtgttctcagctgttctcatgtattagccccacagtactgcagaggcactgaccccactgtttatggggaaatatttaaacactacatgcttgatcattaaaatgagtcagctctcttagtgaaatttcgagcaatcgaataaaagcttgcctattatccttgctgtccaaatacactgatgcttctttttaagtaaaggaaagagaaagggggaagaagcagctactgaggagaaagtgagatttctgtcacatgcatttctccaagaaggaatggttcattgcccgagactcagagttcacacaggcaagtcagctgtggtaggggaaatgcccacttaatagattaaagatattataatagataataatagataaaatagattaatagataaaatagataccaatcttaatagattaaagtgtcctgttaaatataaactgtccacaccatgctgaaatttcctatgccaaatgataccccaccataacagaatgatttctttctggcttcttaccagggatctggttcctacagaaaggtctagaacagctccctctgcacttagaggtccagcgttcatttcatcttagagttaatagtgagttgtgctatctttcatgtggcgggggacttgttgttcactttctgattactttttgagctggaatataagtgctgaagatcaagtgatttaattcccaagccaaatccacatcacaaaacattttgggacagggtttgtaaatatctaaagtgtggagccctgtggtgcttgcacataacgagatggaaagagaacacaaatggggtcctggaaggtacagtaaaacaccctgctgttcttagtcatgtcttgggatggggaatgcttgttttctccaaactaataccaaaggtgtggccactgagcaaccaaatctatgctttctagtctgtgtatactttgaaataaaagggataaaaactacattaggcctatgactagaggccaactttcctgtgattcaattgatagtgtctcccaatgcgcataggcagagacaatgcatagcagtgcatctgaattaaaacctgaatgttccacctattaattacaagcctgagacacactgagg

SEQ ID NO: 24 - DNA, 6625 bp, Artificial Sequence
  transgene with neo excised (wild type)
cgtcgacaagtttgttatgggggaaggttgatagagattaatctaatttaattattttaatataaaaggtaaggatgagaaatagtcttagtaaaatttctaaagtaagggctagtgaaaagtctctcacccagtaagtgcacaccactagcaacatttgttagcattatactgtacacaacatgaaacaaactaactaatgtaccaagcaatttaggaagaagccagtaaatcactaaattacagaacgtttaaaaagtaaactttgtggtatttaaccagcttggtttatatccttgaaaatcaactgggagaaagatactaaaaagcatctaccaggaagtatagaggcaacctgacttgaaagtccttagaaacctcttatcacctttacggagaagcaactcagcttacctgaattgaaccccccaaaccagacttctaagtctttcttctgacccagaatgtgctctctaagaagagacttgacccccattggctctagaagcaactgttaatctcatggttacctgacattttgggcacaacttagagaaaagaatttaaaaatttaaaggcaagcgagggaacagggtctccatagagacagatcgaatgagctccgagaatataaaaagtcgtgctgtttgtagagctatgcataaggtcatgaaagagccaagagtgccctaagtcctcacctttggctgatttctcctctgtgcaaacagagagtgaggggaggggaggctgccaagtgagagtggggtgggggtggggtgacacaatgtaggcttgcttcaacacacaaaactaggagacttcctgtttgtagctgccttcgtccatcactctctgaccagtaagatcacaaatcagatggtttattgggcacatgagacagaccagtacaaacttttctcgtaagtcactgaaataaacaacaatgataaccaaccacggtaacaaaaaaaaaaaaaaatgtaagcagtaaatatggtttccagaatagctgcaacatacttaatatgtccagtttggaacaaaaataaaaagaagtaggaaaggaaataagaaagtatgaccatacacagaagaaaaaaaaaactgacctttaaaagttattcttggggaaagccactgtgtgtgaaattctttaagccagttattatgagcacctcatatatatcacacctataggagtaacattccatcacaggcaattttaataaagcaatataagccttcatgatgctgggttaggaaagactttctttgctatgaaaaccagatgcactctatgcccaagggcaagagttactcagaaagattattcagttaaccacgtgatacttaaacaactccacggagttcttctctatcacttatttactggttagataattcgccattggagaaagtgtgaaaaggaacatgaatatgtaaatcatccttcttaagggctttagtgaagtgtctggagggaacaactgccagctgctgtaacagctgagccacttgggagtttacaatagtaaaagctgccccttgttcctagcgcatttgcggtcgttggtcagctcaagatggaatgagtctagagggggagctctattctgcacagttacttagtagggggcttctacaccacagagcatctagaaactctgcagtcattttccaaacaggagaaaaagtataatcaagagaaaggtgacgcaatgagtaccaactcctgtactattagtcagaattcagtagtaggtcctactcagttcctgagttgcatggcaacactaacttagcacagtgttctgaagaaacggttagcattgtctctaacacacacacacacacacacacacacacacacacacacacacacacacacggggtgcgggggggaatgggggcgggggcggggctcaaagctaagtggccatctcttcaaagcaacacacatgaatgtgcactcactggagctagcctgagtaagtacggagccaaagtgtttacagtttcaggttgtgatgcttccaactatggaagaagaaaaataaacaagttaaaaaacaaaaaccagaacactatctttaaaaaaaaaaacaaacaacaaaacatcaacaaaatcctaccaacctaaccaacaataacaacaacagaccatgcagaatattatcacattgttgatttgtctaagttcacagacaatacgctttcacaaatgccacttgaactttttaaaatacagtgaatgaccacttttccagccgttagagcagaaactgcaaatggagaaacagctaccagcacgcaagaagtgaacctggtcttaatctctggctttgctgtaattctccacagcatactcatctatttaattaaattactgcctgcatttctagtctttcaaactgagccttccgtcattcaggggggcatcctgggcagaggggcagcgcctgcttcgggagccaggacagctgcttttcatttcttttttttatccacaatcacgcatgagttcagtctcaaggaggaaagagcctggctgattcccgaggatttcaggagtatctcagaatgtgctcgagcaatcaagtcatttctcttaaaacttggaaaggaaaatcccgtgataccttcccaccccagcagccctagccactgagccggttgctgcgctgtaatctatctgtgcaagatcgatgtctcagaaccactgaatacggattggtcagaaggaagcagaggaggagccattcagaacaccccccaccccccggcccccctccgtctaaccataagaaatcagcagcccgcactaggctgctctggctcacaaagatgctcaggaagctgaacttctgagaggctgcggtgtgagtgtgggcgcgcagatgcagacgcggctcccagcagtacagcgagtctcagggaaaaggactctgaaaagaccccgagtgaatactaaagtgaaagccgcactgagagagagagaaaaaaaagcaaacagcagactggtcaaccgagaatgagcattcagaagcaccagcttctaaagagacgatgatctgagccgaacccagaagagaccaacaactcctttgcgagttccgctgcttctgaccctgctcctagcaggcgccaagcgcagcctcccaacttctacaggcatacagactgggagagaatcactcggagcttccctgacccaggaggttggatcagttcaaggaggactcaaatccagctgctgcaggaaggatccatgcagaagctgatctcagaggaggacctgaattcggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggcaccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcaggtatccgcggataacttcgtatagcatacattatacgaagttatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtagataacttcgtatagcatacattatacgaagttataagcttgaagttcctatactttctagagaataggaacttcccgggtgggggacagagtgcaaactaggtagatacctgcagactttgtcactctggcccgatctgagcagtgtacttcccaacagctgcctcttctgtgtaatgctttggttgaaaatatctactgtataaatgtaagtttgtgacttttgacatggaaaaaaaagtctcaacgtgttatgtttattgacacgctattttttttgtttgtaaaatgcttatttatgttctatatagtgtgggcgttatgaattgacatgaaagaaaaacagacacccttattaaaactttgacagtgtttctttcctgttatttatcaaggttccacacttgttctttctgtagtggccgaaatcagaaccttattaaacgtgttctcagctgttctcatgtattagccccacagtactgcagaggcactgaccccactgtttatggggaaatatttaaacactacatgcttgatcattaaaatgagtcagctctcttagtgaaatttcgagcaatcgaataaaagcttgcctattatccttgctgtccaaatacactgatgcttctttttaagtaaaggaaagagaaagggggaagaagcagctactgaggagaaagtgagatttctgtcacatgcatttctccaagaaggaatggttcattgcccgagactcagagttcacacaggcaagtcagctgtggtaggggaaatgcccacttaatagattaaagatattataatagataataatagataaaatagattaatagataaaatagataccaatcttaatagattaaagtgtcctgttaaatataaactgtccacaccatgctgaaatttcctatgccaaatgataccccaccataacagaatgatttctttctggcttcttaccagggatctggttcctacagaaaggtctagaacagctccctctgcacttagaggtccagcgttcatttcatcttagagttaatagtgagttgtgctatctttcatgtggcgggggacttgttgttcactttctgattactttttgagctggaatataagtgctgaagatcaagtgatttaattcccaagccaaatccacatcacaaaacattttgggacagggtttgtaaatatctaaagtgtggagccctgtggtgcttgcacataacgagatggaaagagaacacaaatggggtcctggaaggtacagtaaaacaccctgctgttcttagtcatgtcttgggatggggaatgcttgttttctccaaactaataccaaaggtgtggccactgagcaaccaaatctatgctttctagtctgtgtatactttgaaataaaagggataaaaactacattaggcctatgactagaggccaactttcctgtgattcaattgatagtgtctcccaatgcgcataggcagagacaatgcatagcagtgcatctgaattaaaacctgaatgttccacctattaattacaagcctgagacacactgagg

SEQ ID NO: 25 - protein, 331 aa, Artificial Sequence
  R165W MC4R protein
MVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIVITLLNSTDTDAQSFTVNIDNVIDSVICSSLLASICSLLSIAVDRYFTIFYALQYHNIMTVKWVGIIISCIWAACTVSGILFIIYSDSSAVIICLITMFFTMLAMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRY

SEQ ID NO: 26 - protein, 332 aa, Artificial Sequence [sequence omitted]

SEQ ID NO: 27 - protein, 613 aa, Artificial Sequence
  3HA-hMC4R(R165W)-Venus expressed in mouse
MYPYDVPDYAYPYDVPDYAYPYDVPDYADVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIVITLLNSTDTDAQSFTVNIDNVIDSVICSSLLASICSLLSIAVDRYFTIFYALQYHNIMTVKWVGIIISCIWAACTVSGILFIIYSDSSAVIICLITMFFTMLALMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRYPRITSYSIHYTKLSMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 28 - DNA, 1878 bp, Artificial Sequence
  DNA sequence encoding 3HA-hMC4R(R165W)-Venus protein
atgtacccatacgatgttccagattacgcttacccatacgatgttccagattacgcttacccatacgatgttccagattacgctgatgtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagtgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggcaccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcaggtatccgcggataacttcgtatagcatacattatacgaagttatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtagataacttcgtatagcatacattatacgaagttataa

SEQ ID NO: 29 - protein, 596 aa, Artificial Sequence
  myc-hMC4R(WT)-Venus expressed in mouse
MQKLISEEDLNSVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIVITLLNSTDTDAQSFTVNIDNVIDSVICSSLLASICSLLSIAVDRYFTIFYALQYHNIMTVKRVGIIISCIWAACTVSGILFIIYSDSSAVIICLITMFFTMLALMASLYVHMFLMARLHIKRIAVLPGTGAIRQGANMKGAITLTILIGVFVVCWAPFFLHLIFYISCPQNPYCVCFMSHFNLYLILIMCNSIIDPLIYALRSQELRKTFKEIICCYPLGGLCDLSSRYPRITSYSIHYTKLSMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 30 - DNA, 1790 bp, Artificial Sequence
  DNA encoding myc-hMC4R(WT)-Venus expressed in mouse
atgcagaagctgatctcagaggaggacctgaattcggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattgtcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcatcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggcaccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcaggtatccgcggataacttcgtatagcatacattatacgaagttatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagta